(19) United States
(12) Patent Application Publication
(10) Pub. No.: US 2026/0286065 A1
(43) Pub. Date: Sep. 24, 2026

(54) Programming Protein Polymerization with DNA

(71) Applicant: NORTHWESTERN UNIVERSITY, Evanston, IL (US)

(72) Inventors: Chad A. Mirkin, Wilmette, IL (US); Janet R. McMillan, Evanston, IL (US); Oliver G. Hayes, Evanston, IL (US)

(21) Appl. No.: 19/257,019

(22) Filed: Jul. 1, 2025

Related U.S. Application Data
(63) Continuation of application No. 17/275,896, filed on Sep. 13, 2019, now abandoned.
(60) Provisional application No. 62/731,601, filed on Sep. 14, 2018.
(60) Provisional application No. 62/731,735, filed on Sep. 14, 2018.

(51) Int. Cl. C08H 1/00; C07K 14/78; C07K 19/00; C08L 89/00

(52) U.S. Cl. CPC C08H 1/00; C07K 19/00; C08L 89/00

(57) ABSTRACT

The present disclosure is generally directed to methods for making protein polymers. The methods comprise utilizing oligonucleotides for controlling the association pathway of oligonucleotide-functionalized proteins into oligomeric/polymeric materials.

### CROSS-REFERENCE TO RELATED APPLICATIONS

This application claims the priority benefit under 35 U.S.C. § 119(e) of U.S. Provisional Patent Application No. 62/731,601, filed Sep. 14, 2018, and U.S. Provisional Patent Application No. 62/731,735, filed Sep. 14, 2018, each of which is incorporated herein by reference in their entirety.

### STATEMENT OF GOVERNMENT INTEREST

This invention was made with government support under grant number N00014-15-1-0043 awarded by the Office of Naval Research. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2018-151R_Seqlisting.txt”, which was created on Sep. 13, 2019 and is 1,521 bytes in size. The subject matter of the Sequence Listing is incorporated herein in its entirety by reference.

### FIELD OF THE INVENTION

The present disclosure is generally directed to methods for making protein polymers. The methods comprise utilizing oligonucleotides for controlling the association pathway of oligonucleotide-functionalized proteins into oligomeric/polymeric materials.

### BACKGROUND

Supramolecular protein polymers, which are integral to many biological functions, are also important synthetic targets with a wide variety of potential applications in biology, medicine, and catalysis. Polymeric materials formed from the non-covalent association of protein building blocks are supramolecular structures that play critical roles in living systems, guiding motility,¹ recognition, structure, and metabolism.² Supramolecular protein polymers therefore are important synthetic targets with a wide variety of potential applications in biology, medicine, and catalysis. However, with natural biological polymerization events, the organization and reorganization pathways for assembly are carefully orchestrated by a host of complex binding events, which are challenging to mimic in vitro.³⁻⁴ Therefore, while methods have been developed to synthesize protein polymers, the ability to deliberately control the pathways by which they form is not currently possible.⁵⁻⁹

Controlling the polymerization of small molecules, namely via living processes, has revolutionized polymer science by providing synthetic access to complex macromolecules with precisely defined compositions and architectures, and therefore structures with uniform properties and specific functionalities.¹⁰⁻¹² In the field of supramolecular polymerization, recent examples have demonstrated that the conformation or aggregation state of monomers in solution can dictate whether polymerization occurs spontaneously via a step-growth process, or whether an initiation event is first required to overcome a kinetic barrier to polymerization, thereby triggering a chain-growth pathway.¹³⁻¹⁶ Thus, in general, the kinetic barrier towards polymerization, or lack thereof, dictates whether a system follows a spontaneous step-growth pathway, or whether the possibility for chain-growth exists. Despite the large body of literature devoted to honing pathway control over the polymerization of small molecule monomers, the extension of these concepts to building blocks at larger length scales, such as proteins, has not been explored. Indeed, while examples of protein and nanoparticle polymerization by a spontaneous step-growth process have been reported,⁹ the ability to deliberately control the polymerization process of nanoscale building blocks presents a significant challenge due to the inherent difficulties of finely controlling interactions on this length scale.

### SUMMARY

DNA has emerged as a highly tailorable bonding motif for controlling the assembly of nanoscale building blocks, including proteins, into both crystalline and polymeric architectures.¹⁷⁻²³ In these systems, sequence specificity and carefully designed sticky ends, along with ligand placement are employed as design handles to control particle association and therefore the final thermodynamic structure of an assembly. However, in principle, one could use DNA conformation to program the energetic barriers of assembly, and utilize sequence-specific interactions to access such barriers in a manner reminiscent of supramolecular strategies that manipulate polymerization pathways by designing kinetic barriers to polymerization.²⁴

Accordingly, disclosed herein is a strategy that utilizes oligonucleotides for controlling the association pathway of oligonucleotide-functionalized proteins into oligomeric/polymeric materials. Depending on the deliberately controlled sequence and conformation of the appended oligonucleotide, protein-oligonucleotide “monomers” can be polymerized through either a step-growth or chain-growth pathway. The resultant polymers' architecture and distribution were found to be heavily impacted by the association pathway employed. Importantly, in the case of the chain-growth mechanisms, “living” chain ends are also observed. This demonstrates an example of mechanistic control over protein association and establishes a methodology that could be applied to any nanoparticle system. Furthermore, using this strategy, the synthesis of protein oligomers and polymers with complex architectures including sequence-defined, multi-block, brush and branched protein polymer architectures.

Exemplary applications of the subject matter of the disclosure include, but are not limited to:
Multi-step catalysis
Assembly-line biosynthesis
Tissue engineering
Soft-materials with unique bulk physical properties dictated by protein composition

Advantages of the subject matter of the disclosure include, but are not limited to:
Generalizable strategy through which any protein can be incorporated into polymeric structure.
Protein polymer materials with tailorable molecular weight distributions and architecture
Oligonucleotide length can be tailored to define specific inter-protein distance

Accordingly, in some aspects, the disclosure provides a method of making a protein polymer comprising contacting (a) a first protein monomer comprising a first protein to which a first oligonucleotide is attached, the first oligonucleotide comprising a first domain (V) and a second domain (W); and (b) a second protein monomer comprising a second protein to which a second oligonucleotide is attached, the second oligonucleotide comprising a first domain (V′) and a second domain (W′), wherein (i) V is sufficiently complementary to V′ to hybridize under appropriate conditions and (ii) W is sufficiently complementary to W′ to hybridize under appropriate conditions, and wherein the contacting results in V hybridizing to V′, thereby making the protein polymer. In some embodiments, the contacting allows W to hybridize to W′. In some embodiments, the first protein and the second protein are the same. In further embodiments, the first protein and the second protein are different. In some embodiments, the first protein and the second protein are subunits of a multimeric protein. In some embodiments, the first oligonucleotide is attached to the first protein via a lysine or cysteine on the surface of the first protein. In some embodiments, the first oligonucleotide is DNA, RNA, a combination thereof, or a modified form thereof. In further embodiments, V is from about 10-100 nucleotides in length. In some embodiments, W is from about 10-100 nucleotides in length. In some embodiments, the second oligonucleotide is attached to the second protein via a lysine or cysteine on the surface of the second protein. In further embodiments, the second oligonucleotide is DNA, RNA, a combination thereof, or a modified form thereof. In some embodiments, V′ is from about 10-100 nucleotides in length. In some embodiments, W′ is from about 10-100 nucleotides in length. In some embodiments, the protein polymer is a hydrogel or a therapeutic. In further embodiments, the therapeutic is an antibody, a cell penetrating peptide, a viral capsid, an intrinsically disordered protein, a lectin, or a membrane protein.

In some aspects, the disclosure provides a method of making a protein polymer comprising contacting (a) a first protein monomer comprising a first protein to which a first oligonucleotide is attached, the first oligonucleotide comprising a first domain (X), a second domain (Y′), a third domain (Z), and a fourth domain (Y), wherein Y is sufficiently complementary to Y′ to hybridize under appropriate conditions to produce a first hairpin structure; (b) a second protein monomer comprising a second protein to which a second oligonucleotide is attached, the second oligonucleotide comprising a first domain (Y), a second domain (X′), a third domain (Y′), and a fourth domain (Z′), wherein Y is sufficiently complementary to Y′ to hybridize under appropriate conditions to produce a second hairpin structure; and (c) an initiator oligonucleotide comprising a first domain (Y) and a second domain (X′); wherein the contacting results in (i) X′ of the initiator oligonucleotide hybridizing to X of the first oligonucleotide and Y of the initiator oligonucleotide displacing Y of the first oligonucleotide, thereby opening the first hairpin structure and (ii) Z′ of the second oligonucleotide hybridizing to Z of the first oligonucleotide thereby opening the second hairpin structure, and thereby making the protein polymer. In some embodiments, the first protein and the second protein are the same. In some embodiments, the first protein and the second protein are different. In further embodiments, the first protein and the second protein are subunits of a multimeric protein. In some embodiments, the first oligonucleotide is attached to the first protein via a lysine or cysteine on the surface of the first protein. In some embodiments, the first oligonucleotide is DNA, RNA, a combination thereof, or a modified form thereof. In further embodiments, X of the first oligonucleotide is from about 2-20 nucleotides in length. In some embodiments, Y′ of the first oligonucleotide is from about 12-80 nucleotides in length. In some embodiments, Z of the first oligonucleotide is from about 2-20 nucleotides in length. In some embodiments, Y of the first oligonucleotide is from about 12-80 nucleotides in length. In further embodiments, the second oligonucleotide is attached to the second protein via a lysine or cysteine on the surface of the second protein. In still further embodiments, the second oligonucleotide is DNA, RNA, a combination thereof, or a modified form thereof. In some embodiments, Y of the second oligonucleotide is from about 12-80 nucleotides in length. In some embodiments, X′ of the second oligonucleotide is from about 2-20 nucleotides in length. In some embodiments, Y′ of the second polynucleotide is from about 12-80 nucleotides in length. In some embodiments, Z′ of the second polynucleotide is from about 2-20 nucleotides in length. In further embodiments, the protein polymer is a hydrogel or a therapeutic. In various embodiments, the therapeutic is an antibody, a cell penetrating peptide, a viral capsid, an intrinsically disordered protein, a lectin, or a membrane protein. In some embodiments, a method of the disclosure further comprises adding a third protein monomer comprising a third protein to which a third oligonucleotide is attached, the third oligonucleotide comprising a first domain (X), a second domain (Y′), a third domain (Z), and a fourth domain (Y), wherein Y is sufficiently complementary to Y′ to hybridize under appropriate conditions to produce a third hairpin structure. In some embodiments, the third protein is identical to the first protein. In some embodiments, the third protein is identical to the second protein. In some embodiments, a method of the disclosure further comprises adding a fourth protein monomer comprising a fourth protein to which a fourth oligonucleotide is attached, the fourth oligonucleotide comprising a first domain (Y), a second domain (X′), a third domain (Y′), and a fourth domain (Z′), wherein Y is sufficiently complementary to Y′ to hybridize under appropriate conditions to produce a fourth hairpin structure. In some embodiments, the fourth protein is identical to the first protein. In further embodiments, the fourth protein is identical to the second protein. In any of the aspects or embodiments of the disclosure, addition of the third monomer and/or the fourth monomer results in extension of the protein polymer chain. In some embodiments, the amount of initiator oligonucleotide that is added to a reaction is from about 0.2 equivalents to about 1.6 equivalents, or from about 0.2 to about 1.4 equivalents, or from about 0.2 to about 1.2 equivalents, or from about 0.2 to about 1.0 equivalents, or from about 0.2 to about 0.8 equivalents, or from about 0.2 to about 0.6 equivalents, or from about 0.2 to about 0.4 equivalents. In further embodiments, the amount of initiator oligonucleotide that is added to a reaction is, is at least, or is at least about 0.2, 0.4, 0.6, 0.8, 1.0, 1.2, 1.4, 1.6, 1.8, or 2.0 equivalents. In still further embodiments, the amount of initiator oligonucleotide that is added to a reaction is less than or less than about 2.0, 1.8, 1.6, 1.4, 1.2, 1.0, 0.8, 0.6, 0.4, or 0.2 equivalents.

In some aspects, the disclosure provides a method of treating a subject in need thereof, comprising administering a protein polymer of the disclosure to the subject.

In some aspects the disclosure provides a composition comprising a protein polymer of the disclosure and a physiologically acceptable carrier.

### BRIEF DESCRIPTION OF THE FIGURES

FIG. 1 shows a representation of step-growth and chain-growth mGFP-DNA monomer sets. (A) Step-growth monomers SA and SB with a single stranded DNA modification and therefore no kinetic barrier to polymerization. (B) Chain-growth monomers HA and HB possess a hairpin DNA modification, and therefore an insurmountable kinetic barrier to polymerization in the absence of an initiator strand. (C) Proposed association pathways for step-(left) and chain-growth (right) monomer systems based on the DNA sequence design (bottom, boxes). Proposed system free energy diagrams for polymerization events are shown.

FIG. 2 shows a schematic of monomer design. (A) single stranded monomers SA and SB are composed of a set of two DNA strands with a staggered complementary pattern and should polymerize via a step-growth pathway. (B) Hairpin-GFP monomers consist of a set of two hairpin DNA strands HA and HB that cannot assemble in the absence of an initiator strand.

FIG. 3 shows the characterization of GFP-DNA monomers. (A) SDS-PAGE characterization. (B) Analytical size-exclusion characterization showing traces for free DNA (bottom) and protein, as well as protein-DNA conjugates (top).

FIG. 4 shows SEC characterization of polymers. (A) SA+SB, (B) HA+HB with varying concentrations of initiator strand, I.

FIG. 5 shows Cryo-TEM characterization of polymers. Images reveal the formation of both linear and cyclic products of differing DP for step growth monomers, and the formation of only linear products where the DP depends on [I].

FIG. 6 shows that assembly of βGal with DNA on lysine or cysteine residues with complementary AuNPs results in either simple cubic or simple hexagonal arrangement of AuNPs, depending on the chemistry of conjugation. Top: TEM micrographs (Scale bar=500 nm left, and 1 μm right), and bottom: SAXS patterns for resulting AuNP-protein assemblies.

FIG. 7 shows assembly of protein polymers via DNA interactions. (a) Assembly of βGal-DNA mutant into 1D architectures (b) Negative stain TEM characterization of βGal assemblies (Scale bar 200 nm). (c) DNA conformation can dictate protein polymerization pathway. Bottom: cryo-TEM micrograph showing linear and cyclic products for step growth system and linear products only for chain growth system (scale bar 100 nm).

FIG. 8 shows SDS-PAGE characterization of mGFP-DNA monomers. Gel confirms the successful purification of the desired species, and monomer bands display an electrophoretic mobility that corresponds well to the addition of a single oligonucleotide to the surface of the protein. Gel (4-15% TGX, Biorad) was run for 35 minutes at 200 V.

FIG. 9 shows UV-vis spectra of mGFP, free DNA, and DNA-GFP monomers. Each plot shows the spectra for unmodified mGFP (green), free DNA and purified mGFP-DNA conjugates for each monomer. All spectra on each plot are normalized to a concentration of 2 μM and give an approximate ratio of 1 DNA: 1 mGFP for mGFP-DNA conjugates.

FIG. 10 shows SEC chromatograms of native mGFP, free DNA, and mGFP-DNA monomers. Data confirms the absence of free DNA and unconjugated mGFP from purified monomer samples. The chromatogram for mGFP shows a higher molecular weight peak that corresponds to the oxidized dimer of the protein that is removed upon anion exchange purification of the DNA conjugates. mGFP fluorescence and 260 nm absorbance signals are normalized to the same relative ratio on each plot, highlighting the increase in 260 nm absorbance for the mGFP-DNA conjugates compared to free mGFP.

FIG. 11 shows step-growth polymerization of mGFP-DNA monomers, S_{A} and S_{B}. (A) Scheme showing the spontaneous polymerization of single stranded monomers into linear and cyclic products. (B) Cryo-EM micrograph of S_{A} monomer. (C) SEC profiles of S_{A} and S_{B} monomers, and polymerization product after incubation for 24 hours. (D) Cryo-EM micrograph of polymers grown from S_{A} and S_{B} monomers with insets showing dominant cyclic products. Scale bars=50 nm (10 nm in cyclic insets). (E) Histogram of number fraction degree of polymerization of linear (top) and cyclic species (bottom).

FIG. 12 shows a microscopy image of hairpin system with 0.6 equiv. initiator taken at 200 kV without use of the phase plate, representative of the best data acquired.

FIG. 13 shows a microscopy image of hairpin system with 0.6 equiv. initiator taken at 200 kV without use of the phase plate, representative of a typical sample.

FIG. 14 shows representative micrographs and analysis for all samples analyzed by TEM. Left: original image (scale bars=100 nm), Right: analyzed image with fibers traced in blue.

FIG. 15 shows chain-growth polymerization of H_{A} and H_{B} monomers. (A) Scheme showing the initiated polymerization of chain-growth monomers. (B) SEC profiles of H_{A} and H_{B} monomers separately and together after incubation for 24 hours without initiator. (C) Cryo-EM micrograph of H_{A} and H_{B} monomers and insert showing class averaged data. (D) Quantitative analysis of degree of polymerization for monomers with 0.4, 0.6, 0.8, and 1.0 equivalents (equiv.) of initiator (top to bottom). Long dashed lines indicate number average, and short dashed lines indicate weight average degree of polymerization. (E) SEC profiles of chain-growth polymerization products with 0.4, 0.6, 0.8, and 1.0 equivalents of initiator. (F) Cryo-EM micrographs of samples prepared with different concentrations of initiator. (G) Weight and number average degree of polymerization (left axis) and % initial monomer consumption (right axis) as a function of equivalents of initiator added. All scale bars=50 nm.

FIG. 16 shows an SEC chromatogram of H_{A} and H_{B} monomers after incubation for 24 hours, and after 1 week of incubation at room temperature. Chromatograms show no appreciable change between the individual monomers and incubated samples with both monomer types, indicating that the monomers are metastable under the conditions studied. Slight broadening in the peak is attributable to slight degradation in column performance observed at the time of measurement.

FIG. 17 shows the 12 classes that were generated from data processing showing multiple orientations of the protein-hairpin DNA conjugate.

FIG. 18 shows the effect of initiator addition timing on polymer distribution. SEC of H_{A} and H_{B} with 1 equivalent of initiator added over 5 additions at different time intervals. The legend refers to the time interval between each addition: the experiment was conducted by adding 1 equivalent of initiator all at once (0 minutes), or 0.2 equivalents every 5 minutes or 15 minutes until 1 equivalent total had been added to the sample.

FIG. 19 shows SEC chromatograms of DNA only hairpin polymerization. Top to bottom: 1, 0.8, 0.6, 0.4 and 0 equivalents of initiator.

FIG. 20 shows a time course SEC experiment of chain extension polymerization experiment. Polymer sample containing 0.6 equivalents of initiator was prepared under previously described conditions and equilibrated overnight. 50 μL of polymer sample was added to 50 μL of monomer at the same concentration but containing no initiator, immediately prior to injection. SEC injections were performed at 12 minute intervals as previously described.

FIG. 21 shows chain extension of polymers with active chain ends. (A) Scheme showing addition of fresh monomer to sample with active chain ends. (B) Cryo-EM micrograph of resulting chain extension products. (C) Histograms showing an increase in average degree of polymerization for sample before (red) and after (purple) chain extension. Long dashed lines indicate number average, and short dashed lines indicate weight average degree of polymerization. Scale bar=50 nm.

### DETAILED DESCRIPTION

Protein monomer conjugates comprise proteins modified with a single oligonucleotide strand. Based on the sequence of this oligonucleotide strand, it can exist in either a single stranded or hairpin conformation, and these monomers can in some aspects polymerize by a step-growth pathway or chain-growth pathway. This enables control over protein polymer topology (cyclic vs linear) and degree of polymerization.

The terms “polynucleotide” and “oligonucleotide” are interchangeable as used herein.

As used in this specification and the appended claims, the singular forms “a,” “an,” and “the” include plural reference unless the context clearly dictates otherwise.

### Proteins

A “protein” as used herein is understood to include any moiety comprising a string of amino acids. In some embodiments, a protein polymer of the disclosure may be administered to a patient for the treatment or diagnosis of a condition. The term also includes peptides. A “protein monomer” as used herein refers to any protein to which an oligonucleotide is attached and that is able to undergo polymerization according to a method described herein.

Proteins (which include therapeutic proteins) contemplated by the disclosure include, without limitation peptides, enzymes, structural proteins, hormones, receptors and other cellular or circulating proteins as well as fragments and derivatives thereof. Protein therapeutic agents include an antibody, a cell penetrating peptide (for example and without limitation, endo-porter), a viral capsid, an intrinsically disordered protein (for example and without limitation, casein and/or fibrinogen), a lectin (for example and without limitation, concanavalin A), or a membrane protein (for example and without limitation, a receptor, glycophorin, insulin receptor, and/or rhodopsin). Therapeutic agents also include, in various embodiments, a chemotherapeutic agent.

In various aspects, protein therapeutic agents include cytokines or hematopoietic factors including without limitation IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, erythropoietin (EPO), thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor β chain, platelet derived growth factor BB, platelet derived growth factor receptor α, platelet derived growth factor receptor β, pre-B cell growth stimulating factor, stem cell factor receptor, TNF, including TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof. Examples of biologic agents include, but are not limited to, immuno-modulating proteins such as cytokines, monoclonal antibodies against tumor antigens, tumor suppressor genes, and cancer vaccines. Examples of interleukins that may be used in conjunction with the compositions and methods of the present invention include, but are not limited to, interleukin 2 (IL-2), and interleukin 4 (IL-4), interleukin 12 (IL-12). Other immuno-modulating agents other than cytokines include, but are not limited to bacillus Calmette-Guerin, levamisole, and octreotide.

Examples of hormonal agents include, but are not limited to, synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone.

Chemotherapeutic agents contemplated for use include, without limitation, enzymes such as L-asparaginase, biological response modifiers such as interferon-alpha, IL-2, G-CSF and GM-CSF, hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

A protein chemotherapeutic includes an anti-PD-1 antibody.

Structural proteins contemplated by the disclosure include without limitation actin, tubulin, collagen, elastin, myosin, kinesin and dynein.

Hydrogel. In various aspects of the disclosure, the protein polymer is a hydrogel. Protein monomers useful in the production of a hydrogel include, without limitation, structural proteins as described herein (e.g., collagen, elastin, actin), glycoproteins, enzymes, heparin binding protein, fibronectin (cell adhesion), integrin, laminin, proteases, and/or growth factors.

### Modular Protein Architectures

In some aspects, the disclosure provides methods of producing multi-block protein polymers. Such methods take advantage of the “living” character of the protein polymers disclosed herein. The methods of the disclosure provide protein polymers that can continue growing via, e.g., addition of fresh protein monomers to the reaction. Thus, in various embodiments, protein polymers may be synthesized in any combination and portions from multiple different proteins can be combined into a protein polymer. Accordingly, in some embodiments the disclosure contemplates that portions from various proteins are assembled into a single protein polymer (i.e., a heteromeric protein polymer) that exhibits the properties provided by each portion. Alternatively, protein polymers may be synthesized as homopolymers, wherein the protein portion of each protein monomer used to synthesize the protein polymer is the same.

Methods of the disclosure also include those that produce A/B-type structures with alternating proteins along a polymer chain. In some embodiments, chain extension is performed as a function of the living character of these polymers. Protein monomers (either identical to those already polymerized, or different) are added to the pre-polymerized chains which leads to chain extension with the new monomers. In any of the aspects or embodiments of the disclosure, both monomers (e.g., the “first protein monomer comprising a first protein to which a first oligonucleotide is attached” and the “second protein monomer comprising a second protein to which a second oligonucleotide is attached” as described herein) are added for the polymerization to continue. In any of the aspects or embodiments of the disclosure, additional initiator oligonucleotide is added to the reaction.

The amount of initiator oligonucleotide that is added to a reaction is from about 0.2 equivalents to about 2 equivalents. In some embodiments, the amount of initiator oligonucleotide that is added to a reaction is from about 0.2 equivalents to about 1.6 equivalents, or from about 0.2 to about 1.4 equivalents, or from about 0.2 to about 1.2 equivalents, or from about 0.2 to about 1.0 equivalents, or from about 0.2 to about 0.8 equivalents, or from about 0.2 to about 0.6 equivalents, or from about 0.2 to about 0.4 equivalents. In further embodiments, the amount of initiator oligonucleotide that is added to a reaction is, is at least, or is at least about 0.2, 0.4, 0.6, 0.8, 1.0, 1.2, 1.4, 1.6, 1.8, or 2.0 equivalents. In still further embodiments, the amount of initiator oligonucleotide that is added to a reaction is less than or less than about 2.0, 1.8, 1.6, 1.4, 1.2, 1.0, 0.8, 0.6, 0.4, or 0.2 equivalents. As used herein, equivalents of initiator refers to equivalents with respect to a single building block (i.e., protein monomer). For example and without limitation, for 0.4 equiv. initiator, sample contains 0.4 μM initiator, 1 μM of a first protein monomer and 1 μM of a second protein monomer.

### Oligonucleotides

The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′, N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30:613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and International Patent Publication No. WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine (1H-pyrimido[5,4-b][1,4]benzoxazin-2 (3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30:613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594, 121, 5,596,091; 5,614,617; 5,645,985; 5,830,653; 5,763,588; 6,005,096; 5,750,692 and 5,681,941, the disclosures of which are incorporated herein by reference.

Specific examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide.”

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′, or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still other embodiments, oligonucleotide mimetics wherein both one or more sugar and/or one or more internucleotide linkage of the nucleotide units are replaced with “non-naturally occurring” groups. In one aspect, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., 1991, Science, 254:1497-1500, the disclosures of which are herein incorporated by reference.

In still other embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where RH is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H} H—, —NR^{H}—P(O)₂—O—, —O—P(O,NR^{H})—O—, —CH₂—P(O)₂—O—, —O—P(O)₂—CH₂, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H} P(O)₂—O—, —O—P(O,NR^{H})—O—, —O—PO(R″)—O—, —O—PO(CH₃)—O—, and —O—PO(NHR^{N})—O—, where RH is selected form hydrogen and C₁₋₄-alkyl, and R″ is selected from C₁₋₆-alkyl and phenyl, are contemplated. Further illustrative examples are given in Mesmaeker et. al., 1995, Current Opinion in Structural Biology, 5:343-355 and Susan M. Freier and Karl-Heinz Altmann, 1997, Nucleic Acids Research, vol 25: pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, N₃, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In one aspect, a modification includes 2′-methoxyethoxy (2′-O—CH₂CH₂OCH₃, also known as 2′-O-(2-methoxyethyl) or 2′-MOE) (Martin et al., 1995, Helv. Chim. Acta, 78:486-504) i.e., an alkoxyalkoxy group. Other modifications include 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl(2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated herein by reference in their entireties.

In some cases, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Oligonucleotides may also include base modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′: 4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30:613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594, 121, 5,596,091; 5,614,617; 5,645,985; 5,830,653; 5,763,588; 6,005,096; 5,750,692 and 5,681,941, the disclosures of which are incorporated herein by reference.

A “modified base” or other similar term refers to a composition which can pair with a natural base (e.g., adenine, guanine, cytosine, uracil, and/or thymine) and/or can pair with a non-naturally occurring base. In certain aspects, the modified base provides a Tₘ differential of 15, 12, 10, 8, 6, 4, or 2° C. or less. Exemplary modified bases are described in EP 1 072 679 and WO 97/12896.

By “nucleobase” is meant the naturally occurring nucleobases adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴,N⁴-ethanocytosin, N′,N′-ethano-2,6-diaminopurine, 5-methylcytosine (mC), 5-(C³-C⁶)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” thus includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). The term “nucleosidic base” or “base unit” is further intended to include compounds such as heterocyclic compounds that can serve like nucleobases including certain “universal bases” that are not nucleosidic bases in the most classical sense but serve as nucleosidic bases. Especially mentioned as universal bases are 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Proteins of the disclosure to which an oligonucleotide or a modified form thereof is attached generally comprise an oligonucleotide from about 5 nucleotides to about 500 nucleotides in length. More specifically, an oligonucleotide attached to a protein as disclosed herein is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all oligonucleotides intermediate in length of the sizes specifically disclosed to the extent that the oligonucleotide is able to achieve the desired result. Accordingly, in various embodiments an oligonucleotide contemplated by the disclosure is, is at least, or is at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 125, about 150, about 175, about 200, about 250, about 300, about 350, about 400, about 450, about 500 or more nucleotides in length.

Domain. In any of the aspects or embodiments of the disclosure, oligonucleotides comprise one or more domains. As used herein, a “domain” is a nucleotide sequence that is sufficiently complementary to another nucleotide sequence (i.e., another domain) in either the same oligonucleotide or a separate oligonucleotide to allow the two nucleotide sequences (i.e., the two domains) to hybridize. In any of the aspects or embodiments of the disclosure, an oligonucleotide comprises one or more domains. The length of a domain, in various embodiments, is from about 2 to about 20 nucleotides, or from about 10 to about 100 nucleotides, or from about 12 to about 80 nucleotides in length. In further embodiments, the length of a domain is from about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all oligonucleotides intermediate in length of the sizes specifically disclosed to the extent that the oligonucleotide is able to achieve the desired result. In further embodiments, the length of a domain is, is at least, or is at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length. In still further embodiments, the length of a domain is less than or less than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the oligonucleotide attached to a protein is DNA or a modified form thereof. In some embodiments, the oligonucleotide attached to a protein is RNA or a modified form thereof. In some embodiments, the oligonucleotide attached to a protein comprises a sequence (i.e., a domain) that is sufficiently complementary to a domain of a second oligonucleotide attached to a second protein such that hybridization of the oligonucleotide attached to the protein and the second oligonucleotide attached to the second protein takes place, thereby associating the two oligonucleotides. In some embodiments, the oligonucleotide comprises domains that are sufficiently complementary to each other to hybridize, thereby forming a hairpin structure.

In some aspects, multiple oligonucleotides are attached to a protein. In various aspects, the multiple oligonucleotides each have the same sequence, while in other aspects one or more polynucleotides have a different sequence.

Oligonucleotide attachment to a protein. Oligonucleotides contemplated for use in the methods include those bound to a protein or a nanoparticle through any means (e.g., covalent or non-covalent attachment). Regardless of the means by which the oligonucleotide is attached to the protein or nanoparticle, attachment in various aspects is effected through a 5′ linkage, a 3′ linkage, some type of internal linkage, or any combination of these attachments. In some embodiments, the oligonucleotide is covalently attached to a protein or nanoparticle. In further embodiments, the oligonucleotide is non-covalently attached to a protein or nanoparticle.

In some embodiments, an oligonucleotide is attached to a protein in vivo using enzymes. See Bernardinelli et al., Nucleic Acids Research, 2017, Vol. 45, No. 18 e160, incorporated herein by reference in its entirety.

Oligonucleotide complementarity. “Hybridization” means an interaction between two strands of nucleic acids by hydrogen bonds in accordance with the rules of Watson-Crick DNA complementarity, Hoogstein binding, or other sequence-specific binding known in the art. Hybridization can be performed under different stringency conditions known in the art. Under appropriate stringency conditions, hybridization between the two complementary strands could reach about 60% or above, about 70% or above, about 80% or above, about 90% or above, about 95% or above, about 96% or above, about 97% or above, about 98% or above, or about 99% or above in the reactions.

In various aspects, the methods include use of oligonucleotides or domains thereof that are 100% complementary to each other, i.e., a perfect match, while in other aspects, the oligonucleotides or domains thereof are at least (meaning greater than or equal to) about 95% complementary to each other over the relevant length, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to each other over the relevant length. By relevant length is meant the length of an oligonucleotide or a domain thereof that hybridizes to another oligonucleotide or domain thereof as disclosed herein. For example and without limitation, in some aspects of the disclosure, a first oligonucleotide may be 100 nucleotides in length and comprise a domain Y and a domain Y′, wherein domain Y is sufficiently complementary to domain Y′ to hybridize under appropriate conditions; thus if domain Y and Y′ are each 20 nucleotides in length wherein 18 of 20 nucleotides are complementary, then the two domains are 90% complementary to each other.

### Methods of Use/Compositions

In some aspects, the disclosure provides methods of treating a subject in need thereof comprising administering a protein polymer of the disclosure to the subject.

In some aspects, a protein polymer of the disclosure is used in conjunction with one or more nanoparticles (e.g., as exemplified herein) for plasmon enhanced catalytic properties of such materials.

Any protein polymer produced according to the disclosure also is provided in a composition. In this regard, protein polymer is formulated with a physiologically-acceptable (i.e., pharmacologically acceptable) carrier or buffer, as described further herein. Optionally, the protein polymer is in the form of a physiologically acceptable salt, which is encompassed by the disclosure. “Physiologically acceptable salts” means any salts that are pharmaceutically acceptable. Some examples of appropriate salts include acetate, trifluoroacetate, hydrochloride, hydrobromide, sulfate, citrate, tartrate, glycolate, and oxalate. The term “carrier” refers to a vehicle within which the protein polymer is administered to a mammalian subject. The term carrier encompasses diluents, excipients, an adjuvant and a combination thereof. Pharmaceutically acceptable carriers are well known in the art (see, e.g., Remington's Pharmaceutical Sciences by Martin, 1975).

Exemplary “diluents” include sterile liquids such as sterile water, saline solutions, and buffers (e.g., phosphate, tris, borate, succinate, or histidine). Exemplary “excipients” are inert substances include but are not limited to polymers (e.g., polyethylene glycol), carbohydrates (e.g., starch, glucose, lactose, sucrose, or cellulose), and alcohols (e.g., glycerol, sorbitol, or xylitol).

Adjuvants include but are not limited to emulsions, microparticles, immune stimulating complexes (iscoms), LPS, CpG, or MPL.

### EXAMPLES

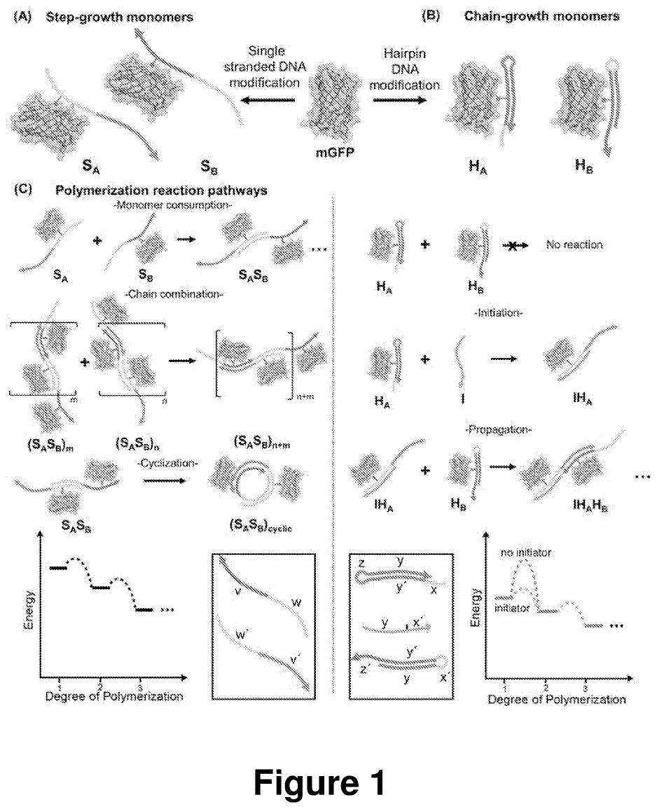

The present disclosure provides methods that utilize oligonucleotides for controlling the polymerization pathway of proteins. We design two sets of mGFP-DNA monomer pairs possessing either a single stranded or hairpin DNA modification and investigate how oligonucleotide sequence can be used to control the polymerization of these two systems (FIG. 1). Characterization of the product distributions using cryo-electron microscopy (Cryo-EM) techniques reveals how the careful design of DNA binding events can program the association of the two monomer sets through either a step-growth or chain-growth pathway in a highly selective and deliberate fashion. Taken together, this work established a general strategy by which the assembly pathway of proteins, or in principle any nanoscale building block, can be finely controlled using oligonucleotide interactions. Importantly, this approach enabled the synthesis of protein polymers with controllable molecular weight distributions and living terminal end groups. This enables the synthesis of protein polymers with precise composition and complex architectures, greatly broadening the scope and functions of such synthetic biomaterials.

### Example 1

Synthesis and characterization of protein-DNA monomers. GFP was expressed in a bacterial expression system, and purified using Ni-NTA affinity. DNA was synthesized using standard solid-phase protocols with reagents purchased from Glen Research. The following sequences were employed:

| | | SEQ |
|---|---|---|
| | | ID |
| Name | Sequence (5′→3′) | NO: |
| H_{A} | TTAACCCACGCCGAATCCTAGACTCAAAGTAGTCTAG | 1 |
| | GAT NH2 TCGGCGTG | |
| H_{B} | AGTCTAGGATT NH2 CGGCGTGGGTT | 2 |
| | AACACGCCGAACCAGACTACTTTG | |
| I | AGTCTAGGATTCGGCGTGGGTTAA | 3 |
| S_{A} | TTAGTCGTCTCTCATCATGTGTTACAAAGTAGTCTAG | 4 |
| | GAT NH2 TCGGCGTG | |
| S_{B} | TAACACATGAT NH2 GAGAGACGACT AA | 5 |
| | CACGCCGAATCCTAGACTACTTTG | |

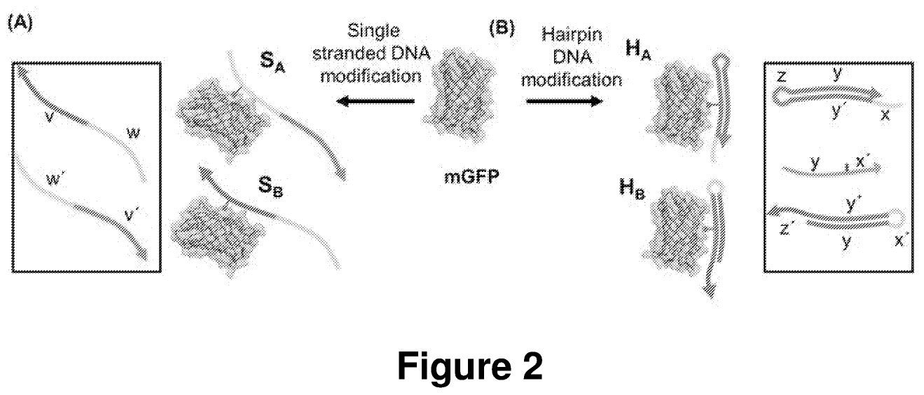

DNA was conjugated to the surface thiol of GFP using pyridyl disulfide chemistry by adding a ten-fold excess of pyridyl disulfide terminated DNA (prepared by reaction amino-DNA with succinimidyl 3-(2-pyridyldithio) propionate cross linker). Reactions were purified via consecutive Ni-NTA affinity and anion exchange to yield protein monomers with single DNA modifications, as revealed by SDS-PAGE and size exclusion chromatography characterization (FIG. 2). UV-vis spectra of the conjugates also support the successful conjugation and purification, where the absorbance at 260 nm of GFP-DNA conjugates is significantly elevated compared to free DNA.

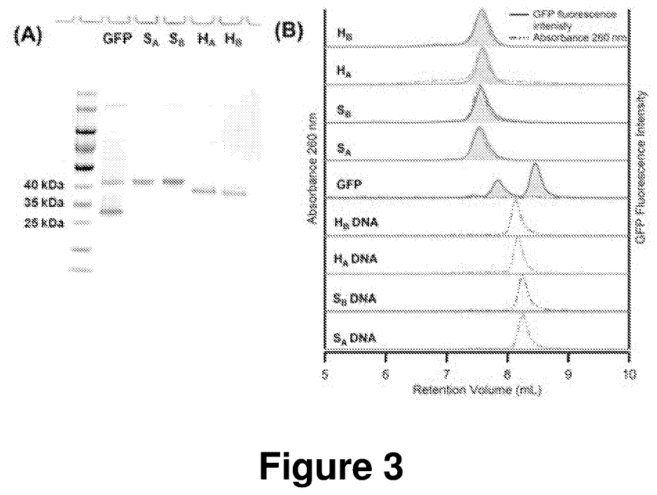

Assembly of protein-DNA polymers. Protein polymers were assembled by combining A and B monomer types in equimolar ratios at room temperature in 1×PBS+0.5 M NaCl followed by overnight incubation. GFP-DNA monomers were analyzed by SDS-PAGE and analytical size-exclusion characterization (FIG. 3).

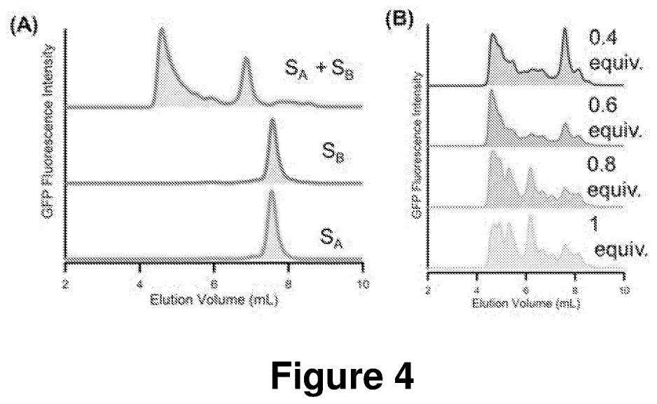

Characterization of protein-DNA polymers via SEC and Cryo-TEM. Polymers were characterized by analytical SEC using an Agilent 1260 Infinity HPLC equipped with an Advanced Bio SEC 300 Å column (Agilent). Results showed a dependence of product distribution on initiator concentration (FIG. 4).

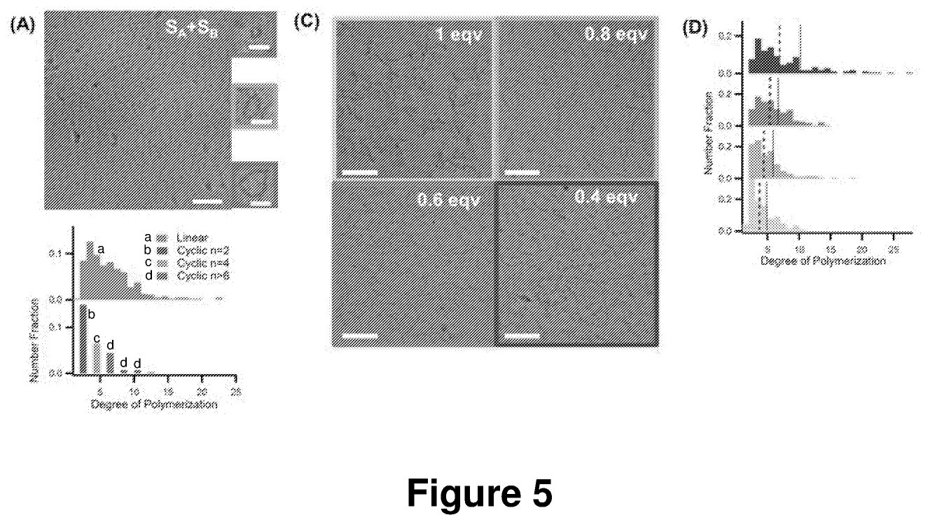

Cryo-TEM characterization was conducted by vitrifying samples using a Mark IV vitrobot on holey carbon TEM grids. Images were collected on a JEOL 3200FS equipped with a Volta phase plate and a K2 summit camera (Gatan). Images of structures showed clear assembly into 1D polymeric materials, and allowed the molecular weight distributions to be estimated. This confirmed the dependence of degree of polymerization on initiator concentration for the hairpin system, and showed a distribution of cyclic and linear products for the single stranded DNA system (FIG. 5).

### Example 2

Proteins are the central building blocks of biological systems, and are powerful synthons for supramolecular materials because of their well-defined structures and sophisticated chemical functions. Their assembly into well-defined 1, 2 and 3D functional structures in nature has inspired efforts to engineer the assembly of proteins into designed architectures [Pieters et al., J., Natural supramolecular protein assemblies. Chem. Soc. Rev. 2016, 45 (1), 24-39; Mann, Angew. Chem. Int. Ed. 2008, 47 (29), 5306-5320]. The assembly of proteins, however, is difficult to control synthetically owning to the chemical heterogeneity of their surfaces, representing a major challenge towards this goal [Papapostolou et al., Mol. Biosyst. 2009, 5 (7), 723-732]. To address this challenge, the present example investigated the use of DNA interactions, which are robust and programmable [Jones et al., Science 2015, 347 (6224)], to mediate the assembly of proteins, and developed a fundamental understanding of how DNA modifications on the surfaces of proteins can be designed to control assembly outcome.

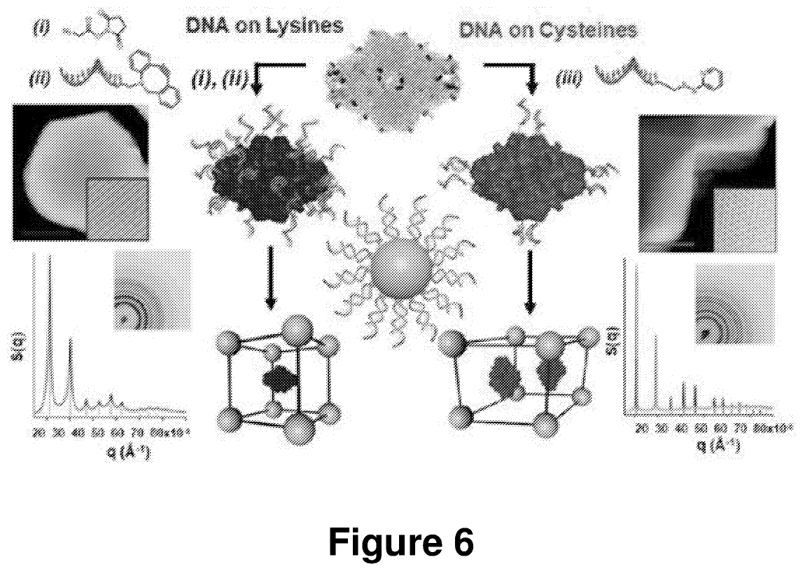

To append DNA to the surface of proteins, surface amines (lysines) or thiols (cysteines), can be selectively reacted with oligonucleotides through either reaction with an NHS-ester-azide crosslinker and cyclooctyne-terminated DNA, or reaction with pyridyl disulfide terminated DNA (FIG. 6). A key challenge that first addressed was the development of a robust analytical strategy to characterize proteins with surface DNA modifications (protein-DNA conjugates). Absorbance spectroscopy, mass spectrometry (MALDI-TOF) and denaturing polyacrylamide gel electrophoresis (SDS-PAGE) determine the protein: DNA ratio in solution, and whether the DNA is covalently conjugated versus non-specifically adsorbed to the protein. Circular dichroism ensures the conformation of the protein is not disrupted by modification, and size exclusion chromatography (SEC) enables the hydrodynamic size of the conjugates to be assessed.

Because of the chemical heterogeneity of protein surfaces, amine and thiol groups are often presented with drastically different spatial distributions. Therefore, the chemistry of DNA conjugation will change both the number and position of DNA modifications, prompting the question: can the chemistry of conjugation, and therefore its spatial distribution of the DNA on protein surfaces affect assembly outcome? To answer this question, protein-DNA conjugates were prepared using the enzyme beta-galactosidase (βGal), which has 36 evenly distributed lysine residues compared to 8 cysteine residues localized at the corners of the protein, by separately functionalizing each residue with DNA. These two conjugates were then co-assembled with gold nanoparticles (AuNPs) functionalized with a complementary oligonucleotide sequence to probe their assembly properties, since AuNP-based crystalline assemblies can be easily characterized. Small-angle X-Ray scattering (SAXS) and TEM characterization revealed that the chemistry of DNA conjugation altered the favored arrangement of AuNPs around the protein: while lysine-functionalized βGal resulted in a simple-cubic nanoparticle arrangement, cysteine-functionalized βGal favored a simple-hexagonal AuNP arrangement (FIG. 6) [McMillan et al., J. Am. Chem. Soc. 2017, 139 (5), 1754-1757].

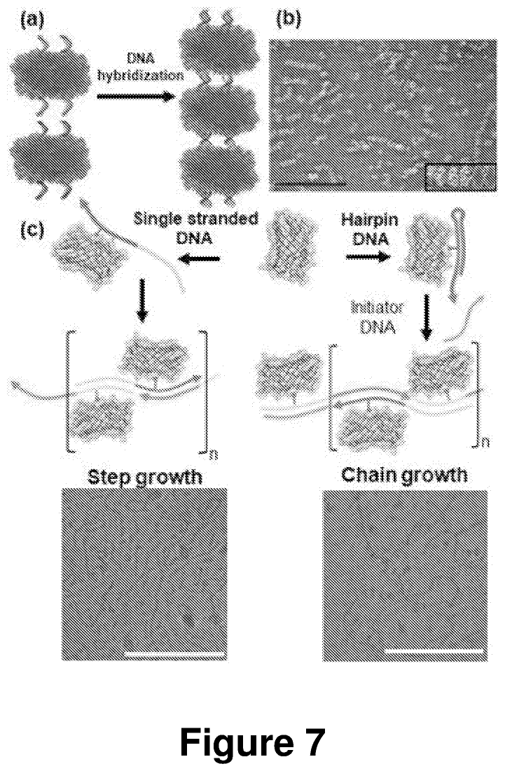

This fundamental observation that the placement of DNA can alter protein assembly led to exploring whether it was possible to access other classes of protein structures, such as one-dimensional (1D) materials, by rationally controlling the placement of DNA modification sites. To do this, the protein sequence of Gal was altered using site-directed mutagenesis techniques, such that pairs of closely positioned thiol groups were located exclusively on the top and bottom face of the protein. Functionalization of this protein resulted in a conjugate with precisely four DNA modifications, and temperature-dependent association studies of complementary building blocks provided strong evidence that proteins interacted in a face-to-face manner (FIG. 7a). Characterization of these assemblies with both negative-stain and cryo-TEM demonstrated the formation of 1D protein structures mediated by DNA-interactions (FIG. 7b) [McMillan et al., J. Am. Chem. Soc. 2018, 140 (22), 6776-6779].

1D protein assemblies are important materials for a host of biocatalysis applications, however, in contrast to molecular-scale monomers, it is not possible to control their mechanism of formation, which greatly inhibits control over their molecular weight and architecture. With DNA, however, the energy barrier towards polymerization can be finely controlled through its sequence and therefore conformation, presenting the possibility of designing both step- and chain-growth assembly pathways. To do this, two sets of protein building blocks functionalized with either a single-stranded or hairpin DNA that is designed to polymerize by either a step- or chain-growth mechanism was synthesized and characterized. Characterization of these systems with both SEC and cryo-TEM provided strong evidence for the difference in polymerization pathway, namely the observation of cyclic and linear product distributions for the step-growth system, and exclusively linear products with a degree of polymerization dependent on initiator concentration for the chain-growth system (FIG. 7c). This work represented the first example where the pathway of protein polymerization (or any nanoscale building block) can be rationally controlled, and the first instance of synthetic control over the molecular weight of protein polymers. Further, this work enables the synthesis of currently inaccessible protein architectures such as block or brush protein polymers.

Overall, the example demonstrated a fundamentally new strategy to assemble proteins into well-defined architectures, and shown that conjugation chemistry, protein sequence, and the conformation of DNA are important design parameters in determining both the final thermodynamic assembly, and the pathway of assembly in these systems. Taken together, this work has overcome a major challenge in the field of protein assembly in trading chemically complex protein-protein interactions with highly modular DNA interactions, which will enable the synthesis of currently inaccessible protein architectures with applications in catalysis and tissue engineering.

### Example 3

As described herein, in any of the aspects of the disclosure, methods are provided that utilize oligonucleotides for controlling the association pathway of proteins. In some aspects, the methods comprise use of sequence-specific oligonucleotide interactions to program energy barriers for polymerization, allowing for either step-growth or chain-growth pathways to be accessed. Two sets of mutant green fluorescent protein (mGFP)-DNA monomers with single DNA modifications were synthesized and characterized. Depending on the deliberately controlled sequence and conformation of the appended DNA, these monomers can be polymerized through either a step-growth or chain-growth pathway. Cryo-electron microscopy with Volta phase plate technology enables the visualization of the distribution of the oligomer and polymer products, and even the small mGFP-DNA monomers. Whereas cyclic and linear polymer distributions were observed for the step-growth DNA design, in the case of the chain-growth system, linear chains were exclusively observed, and a dependence of the chain length on the concentration of initiator strand was noted. Importantly, the chain-growth system possesses a living character, whereby chains can be extended with the addition of fresh monomer. This work represents an important and early example of mechanistic control over protein assembly, thereby establishing a robust methodology for synthesizing oligomeric and polymeric protein-based materials with exceptional control over architecture.

Oligonucleotide design, synthesis and purification. Oligonucleotides were synthesized on solid supports using reagents obtained from Glen Research and standard protocols. Products were cleaved from the solid support using 30% NH₃ (aq) for 16 hours at room temperature, and purified using reverse-phase HPLC with a gradient of 0 to 75% acetonitrile in triethylammonium acetate buffer over 45 minutes. After HPLC purification, the final dimethoxytrityl group was removed in 20% acetic acid for 2 hours, followed by an extraction in ethylacetate. The masses of the oligonucleotides were confirmed using matrix-assisted laser desorption ionization mass spectrometry (MALDI-MS) using 3-hydroxypicolinic acid as a matrix.

For the chain-growth system, previously reported hairpin sequences were employed [Dirks et al., PNAS 2004, 101 (43), 15275-15278]. In the case of the step-growth system, sequences were designed using the IDT oligoanlayzer tool, where the sequence of a single domain was iterated until the sequence afforded no secondary structure elements that displayed a predicted melting temperature above 25° C.

TABLE 1
DNA sequences, molecular weights, and extinction coefficients.

| | | SEQ | MW | MW | |
|---|---|---|---|---|---|
| | | ID | expected | observed | |
| Name | Sequence (5′→3′) | NO: | (Da) | (Da) | _{ϵ260} M⁻¹cm⁻¹) |
| H_{A} | TTAACCCACGCCGAATCCTAGACTCA | 1 | 14890 | 14811 | 463800 |
| | AAGTAGTCTAGGAT NH₂TCGGCGTG | | | | |
| H_{B} | AGTCTAGGATT NH₂CGGCGTGGGTT | 2 | 14953 | 14982 | 461500 |
| | AACACGCCGAACCAGACTACTTTG | | | | |
| I | AGTCTAGGATTCGGCGTGGGTTAA | 3 | 7464 | 7444 | 239600 |
| S_{A} | TTAGTCGTCTCTCATCATGTGTTACAA | 4 | 14949 | 14960 | 461700 |
| | AGTAGTCTAGGAT NH₂TCGGCGTG | | | | |
| S_{B} | TAACACATGAT NH₂GAGAGACGACT | 5 | 14892 | 14845 | 476300 |
| | AA CACGCCGAATCCTAGACTACTTTG | | | | |

T NH₂ = C6 Amino dT modifier from Glen Research

Synthesis and Characterization of mGFP-DNA Monomers

mGFP expression and purification. The mutated plasmid containing the gene for the mutated EGFP (mGFP) that has been previously described was transformed into One Shot®BL21 (DE3) Chemically Competent E. coli (Thermo Fisher) by heat shock, and cells were grown overnight on LB Agar plates with 100 μg/mL ampicillin. Single colonies were picked, and 7 mL cultures were grown overnight at 37° C. in LB broth with 100 μg/mL Ampicillin [Hayes et al., J. Am. Chem. Soc. 2018, 140 (29), 9269-9274]. These cultures were added to 1 L of Terrific Broth (Thermo Fisher) with 1% glycerol and 100 μg/mL ampicillin, and cells were grown at 37° C. to an optical density of 0.6, then induced with 0.02 wt % arabinose overnight at 17° C. Cells were spun down (6000 g, 30 minutes) and resuspended in 100 mL of 1× PBS, then lysed using a high-pressure homogenizer. The cell lysate was clarified by centrifugation at 30 000 g for 30 minutes and loaded onto a Bio-Scale™ Mini Profinity™ IMAC Cartridge (Bio-Rad). The column was washed with 100 ml of resuspension buffer, then eluted in the same buffer with 250 mM imidazole. The eluted fraction was further purified by loading on to Macrp-Prep® DEAE Resin, and washing with 20 mL of 1×PBS. mGFP was eluted with a solution of 1×PBS+0.25 M NaCl.

DNA conjugation. DNA conjugation was carried out immediately after purification using a previously described method [Hayes et al., J. Am. Chem. Soc. 2018, 140 (29), 9269-9274]. Briefly, amine terminated DNA (300 nmoles) was reacted with 50 equivalents of SPDP (Thermo Fischer Scientific) crosslinker in 50% DMF, 1× PBS+1 mM EDTA for 1 hour at room temperature. Excess SPDP was removed from the DNA by two rounds of size exclusion using NAP10 and NAP25 columns (GE Healthcare) equilibrated with PBS (pH 7.4), consecutively. Ten equivalents of the resulting pyridyl disulfide terminated DNA was added to 1.5 ml of 20 μM protein solution, and the reaction allowed to proceed for 16 hours at room temperature. For hairpin DNA-mGFP conjugation reactions, hairpin DNA was snap cooled after SPDP conjugation, but before being added to mGFP. This consisted of heating the DNA solutions to 95° C. for 4 minutes, then 3 minutes at 4° C. The DNA solutions were then equilibrated at room temperature for 5 minutes before adding to the protein solution.

Purification and characterization of mGFP-DNA monomers. mGFP-DNA monomers were purified using a two-step protocol to ensure removal of both unreacted DNA and protein. First, samples were loaded on Ni-NTA column, and washed with 30 mL of 1×PBS to ensure removal of excess DNA. The protein sample was then eluted with a solution of 1×PBS+250 mM imidazole. This eluent was then loaded on Macro-Prep® DEAE Resin, and washed with 20 mLs of 1×PBS, and 1×PBS+0.25 M NaCl. Subsequently, mGFP-DNA conjugates were eluted with a solution of 1×PBS+0.5 M NaCl, and analyzed via SDS-PAGE to ensure successful DNA conjugation and purification.

Size exclusion characterization. Size-exclusion chromatograms were collected using an Agilent 1260 Infinity HPLC equipped with an Advanced Bio SEC 300 Å column (Agilent). All chromatograms reported in this work were monitored at 260 nm, and using a fluorescence detector with an excitation at 488 nm and an emission of 520 nm. Samples were measured with an injection volume of 5 μL at a flow rate of 1 mL/min. For monomer characterization, samples were injected at concentrations between 2 and 5 μM. For polymer characterization, samples were injected at the concentration of assembly.

### Polymer Assembly

Polymer assembly conditions. All mGFP-DNA polymers studied were assembled at 1 μM of each building block (2 μM total protein concentration) in 1×PBS+0.5 M NaCl at room temperature. For all characterization data presented, samples were incubated for a minimum of 12 hours at room temperature prior to analysis. For the chain-growth system, both monomers were combined and mixed in solution prior to the addition of the initiator strand. In this system, equivalents of initiator reported refer to equivalents with respect to a single building block (e.g., for 0.4 equiv. initiator, sample contains 0.4 μM initiator, 1 μM H_{A} and 1 μM H_{B}.).

Polymerization kinetics measurements. Kinetic measurements were conducted by adding initiator to a sample immediately (approximately 15 seconds) prior to SEC injection, and calculating the integrated area percent of the monomer peak after this first injection as an estimate of the initial rate of polymerization. The error bars reported herein report the standard deviation from triplicate measurements.

### Cryo-TEM Imaging

Sample freezing and imaging. Sample solutions were deposited onto 400 mesh 1.2/1.3 C-Flat grids (Protochips) and were plunge frozen into liquid ethane using a Vitrobot™ Mark IV. The grids were imaged using a JEOL 3200FS microscope operating at 300 kV equipped with a Volta phase plate and Omega energy filter. The microscope was aligned and adjusted to give 90° phase shift in acquired images. Movies were acquired on a K2 summit camera (Gatan) with a defocus range between 0.1-1.0 μm using counting mode with a pixel size of 1.1 Angstrom. The dose rate that was used was approximately 10e-/pix/s (equivalent to 8.3e-/Å²/s on the plane of the sample) for a total exposure of 6 seconds.

Data acquisition and class average data processing. 12 recorded movies were subjected to motion correction with MotionCor2 [Zheng et al., Nature Methods 2017, 14, 331]. Following CTF estimation with CTFFIND4 [Rohou et al., Journal of Structural Biology 2015, 192 (2), 216-221], 8 micrographs with the best quality were then selected for further processing. Approximately 1500 particles were picked with a box size of 96 Angstroms, extracted, and 2D classification was all done within RELION-2 software package [Kimanius et al., eLife 2016, 5, e18722].

Analysis of polymer length distributions. Polymer lengths were analyzed using FiberApp [Usov et al., Macromolecules 2015, 48 (5), 1269-1280]. The relatively large noise level in the images necessitated that the polymers be identified visually. Only fibers where clear beginning and end points could be identified were counted, and every identifiable fiber was counted in each image analyzed. Images were binned and inverted prior to analysis in FiberApp to make fibers easier to visualize. For all samples 2-3 images were analyzed to give polymer number counts greater than 200. The calculated length generated by FiberApp was then converted to degree of polymerization (DP) using the following conversion based on the rise-per-base pair of double stranded DNA and then rounded to the nearest whole number:

$$D P = \frac{length ( nm )}{24 bp \times 0.332 nm / bp} \qquad ( 1 )$$

Monomer design and synthesis. To direct the pathway of DNA-mediated protein polymerization, two distinct sets of DNA sequences were designed that, although identical in their overall complementarity, differ in the energy barrier that exists for polymerization. The DNA design for protein monomers expected to engage in a step-growth process (FIG. 1A), consists of two 48 base pair (bp) strands that possess minimal secondary structure, and therefore a minimal energetic barrier for monomer association. Polymerization of the step-growth monomers is driven by the staggered complementary overlap between two halves of each of the 48 bp DNA sequences. Therefore, the indefinite association of alternating A and B strands in one dimension is possible. To realize a chain growth polymerization pathway (FIG. 1B), DNA sequences where monomers would remain kinetically trapped until the addition of an initiator sequence were utilized. To this end, the hybridization chain reaction, a DNA reaction scheme where a set of two hairpins can be induced to polymerize upon the addition of an initiator sequence, was employed.²⁴ Here, two 48 bp hairpins were used, with a 18 bp stem and orthogonal 6 bp toeholds such that the loop of hairpin A was complementary to the toehold of hairpin B. Polymerization will only occur when an initiator strand opens hairpin A, thereby exposing its loop sequence that is complementary to the toehold of hairpin B, thus inducing a cascade of hairpin opening. Overall, each set of DNA sequences employed possesses an identical length and duplexation pattern, with 65% of A- and B-type sequences being identical between step- and chain-growth DNA (Table 1). They differ, however, in the designed conformation and conditions required to initiate polymerization.

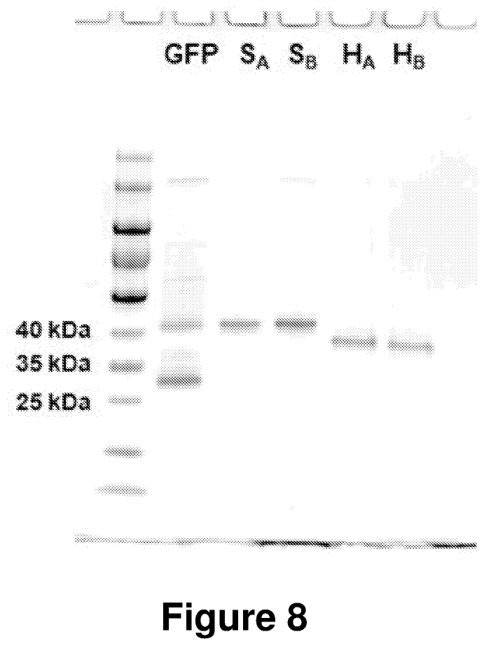

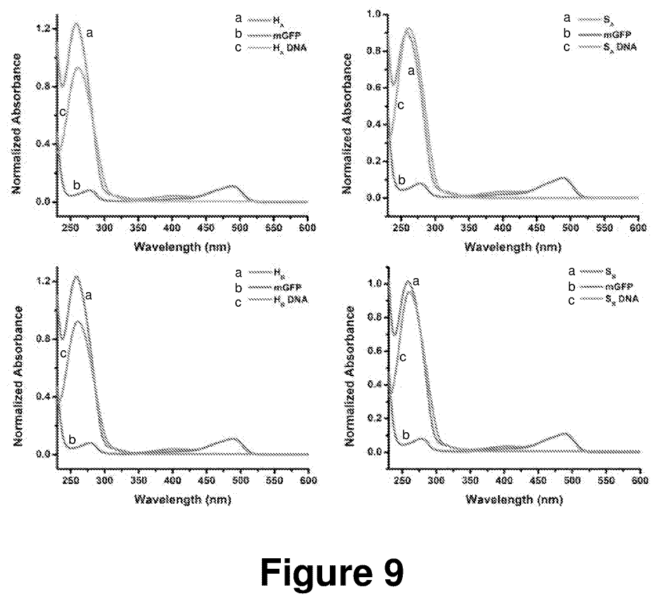

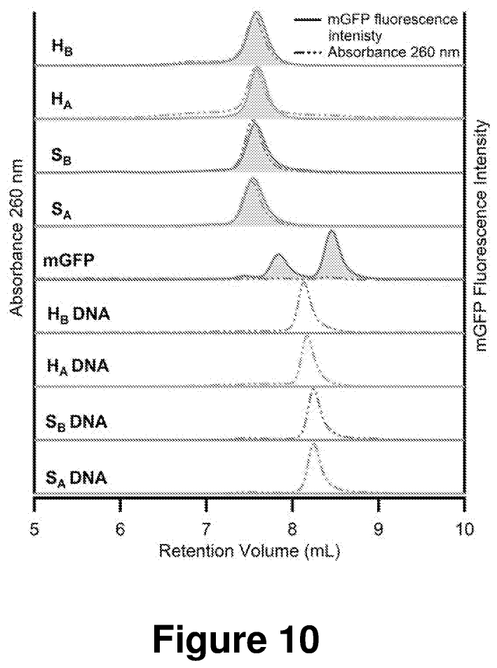

A mutant, green fluorescent protein (mGFP) was chosen as a model system to explore how DNA sequence can be used to program the polymerization pathway of protein monomers. Its monomeric oligomerization state and solvent accessible cysteine residue (C148) enable the preparation of protein-DNA conjugates with a single modification of the designed oligonucleotides. For all the systems studied, mGFP-DNA monomers were prepared by adapting previously published procedures (see hereinabove for description).²³ Briefly, an excess of pyridyl disulfide-functionalized oligonucleotide was incubated with mGFP overnight, followed by purification by anion-exchange to remove any unreacted protein, and nickel-affinity to remove excess DNA. SDS-PAGE analysis of both the single stranded protein-DNA conjugates, S_{A} and S_{B}, and the hairpin protein-DNA conjugates, H_{A} and H_{B}, revealed single protein bands with a decrease in electrophoretic mobility, consistent with the incorporation of a single 48 bp DNA modification (FIG. 8). Importantly, both H_{A} and H_{B} displayed slightly higher mobilities than S_{A} and S_{B}, consistent with the more compact DNA conformation resulting from the hairpin sequences employed. In addition, UV-vis spectra of the conjugates revealed ratios of mGFP chromophore absorbance (488 nm) to DNA absorbance (260 nm) that were consistent with the conjugation of a single strand of DNA to each protein (FIG. 9). Finally, analytical size-exclusion chromatography (SEC) of all monomers showed discrete peaks that confirmed the expected mass increase, as well as the absence of any free DNA or aggregated protein (FIG. 10). Taken together, these data unambiguously confirmed the synthesis and purification of the desired protein-DNA conjugates. Significantly, each set of monomers synthesized are nearly identical in their overall mass, and the appended DNA strands possess identical staggered complementarity between A and B monomers, differing only in the conformation of the DNA modification. One conclusion that came out of this work, therefore, was that this small difference in sequence, and thereby conformation of the protein-appended DNA alters the underlying pathway of polymerization of the monomers between a spontaneous, step-growth process, to an initiated, chain-growth one.

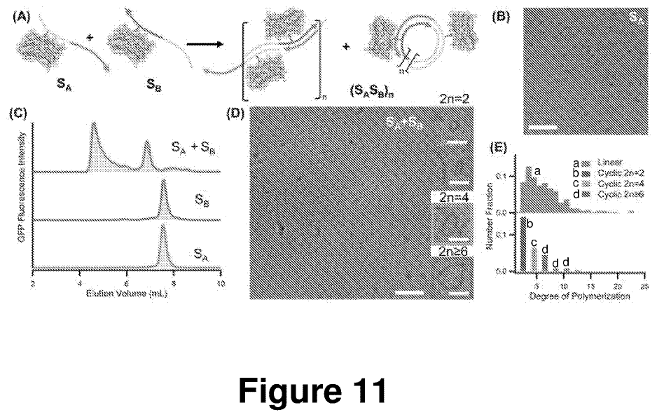

Step-growth polymerization. We first studied the polymerization of single stranded mGFP-DNA monomers using analytical SEC as an effective method of characterizing the aggregation state of mGFP. The combination and overnight incubation of equimolar amounts of S_{A} and S_{B} monomers at room temperature resulted in size exclusion profiles indicative of near complete monomer consumption, and the presence of higher-order aggregates (FIG. 11C). While the majority of species in solution were above the exclusion limit of the column employed, low molecular weight species were also present. The lower molecular weight species that persisted in the sample, even after several days, suggested the presence of cyclic products.

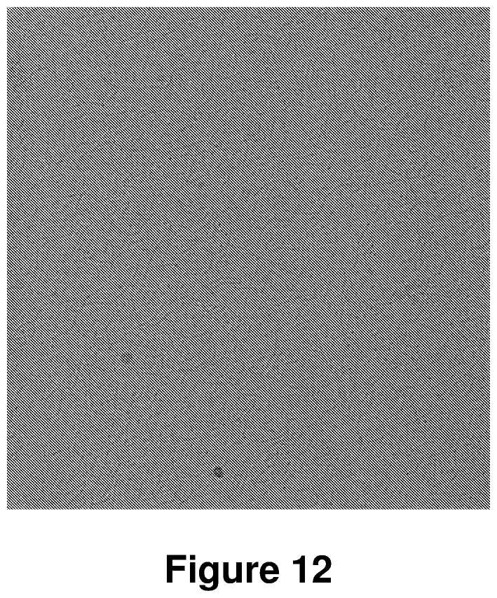

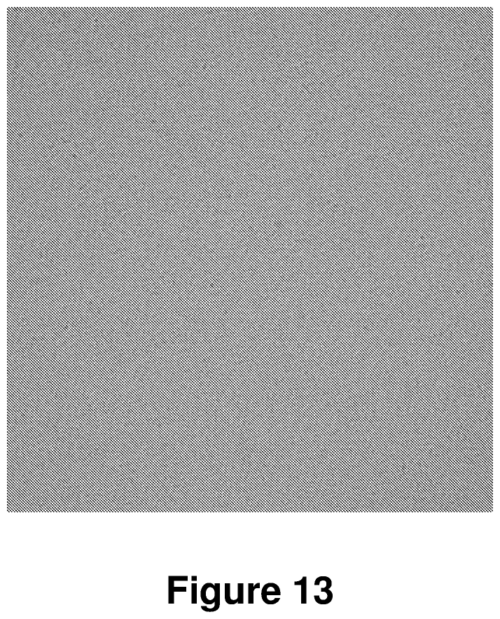

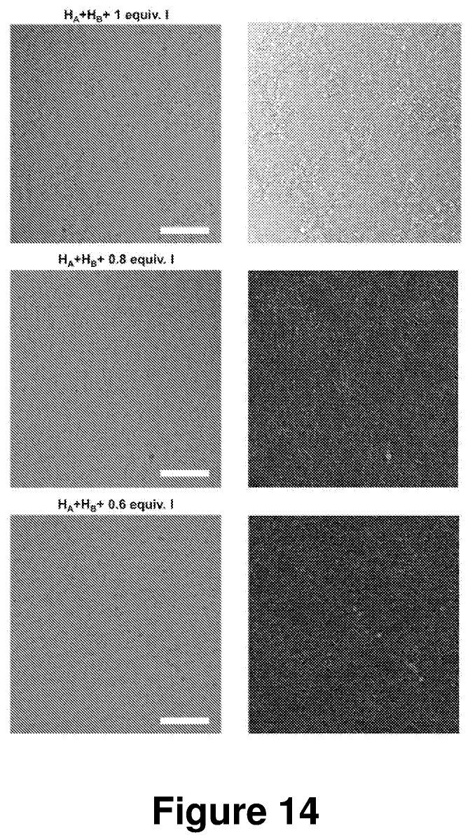

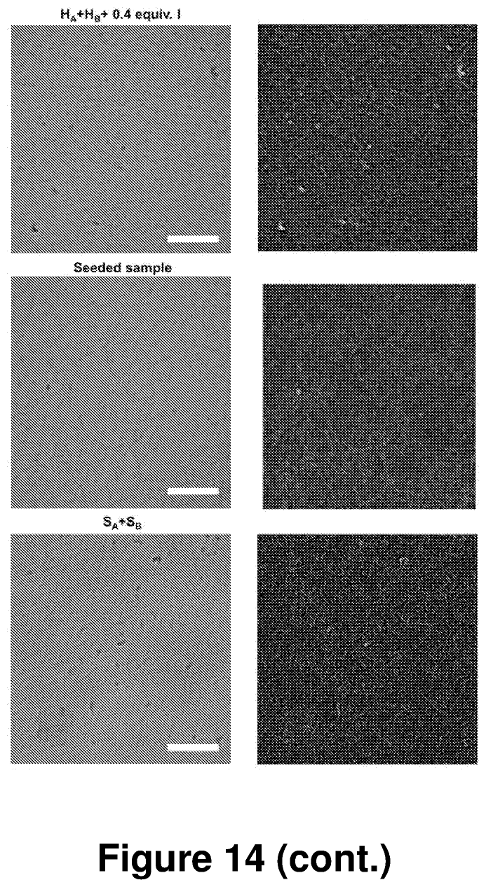

To better characterize the product distribution, the samples were analyzed by cryo-EM to enable the direct characterization and quantification of product distribution, including possible cyclic products. Obtaining images with sufficient contrast to enable the conclusive identification of species composed of mGFP monomers, a protein much smaller than those routinely visualized via cryo-EM, connected through a double stranded DNA backbone is nontrivial. Indeed, even when employing large defocus with a direct-electron detector camera, the synthesized structures could barely be discerned (FIGS. 12, 13). To improve the contrast in these images, a Volta phase plate was employed, a thin continuous carbon film which phase shifts the scattered electron beam, increasing in-focus phase contrast, and thereby greatly enhancing the signal-to-noise ratio in the images.²⁵⁻²⁷ The phase plate enabled the double stranded DNA backbone to be clearly visualized, and in certain images, small spots of electron density corresponding to mGFP could also be visualized (FIG. 11B, 11D). The micrographs clearly revealed a mixture of linear and cyclic products, which were quantified using a fiber analysis software (FIG. 14).²⁸ This analysis revealed that cyclic products, formed through intra-chain hybridization of terminal complementary overhangs, accounted for 28 number percent of the overall product distribution. Quantification of cycle circumference enabled us to determine that the dominant cyclic product formed (15 number percent) is through the dimerization of S_{A} and S_{B}.

Cyclic oligomers are a commonly observed side product of both covalent and supramolecular polymerizations that undergo a step-growth mechanism, where both ends of a growing polymer chain are reactive, and therefore the possibility of cyclization exists. Indeed, the presence of cyclic products has been posited in DNA-only polymerization systems with similar staggered DNA designs but have never been observed directly.²⁹ The observed distribution of cyclic products, dominated by a 48 bp cyclic dimer having a 15 nm diameter may appear surprising at first given the widely reported persistence length of DNA of approximately 50 nm.³⁰⁻³² However, the bending of double stranded DNA well below its persistence length has been reported: DNA as short as 63 bps in length has been shown to form cyclic structures spontaneously for double strands containing a ten-bp single stranded overhang region that hybridizes upon cyclization (compared to 24 bps in this system),³³⁻³⁵ and template-directed ligation approaches have been reported to result in un-nicked cycles as small as 42 bps.³⁶ Furthermore, sharply bent DNA can be explained by the presence of kinks,³⁷ which form at DNA nick sites.³⁸ Interestingly, cyclic dimers can be observed with both circular conformations, and more oblate conformations, where it appears that sharp DNA bending may be occurring at nick sites (FIG. 11D).

The cryo-EM techniques employed have enabled the thorough characterization of products resulting from the mGFP monomers with single stranded DNA modifications, demonstrating a distribution consistent with the designed step-growth formation process. This EM study also suggested that cryo-EM coupled with phase plate technology is a powerful platform to readily observe the conformations of sharply bent DNA, and lend insight into the topology of small DNA minicircles.³⁹

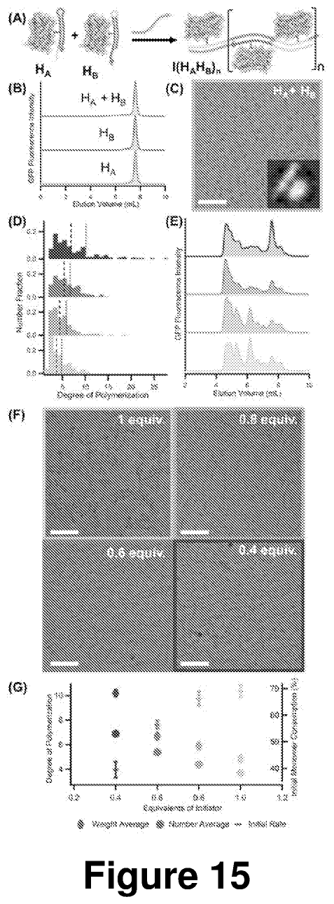

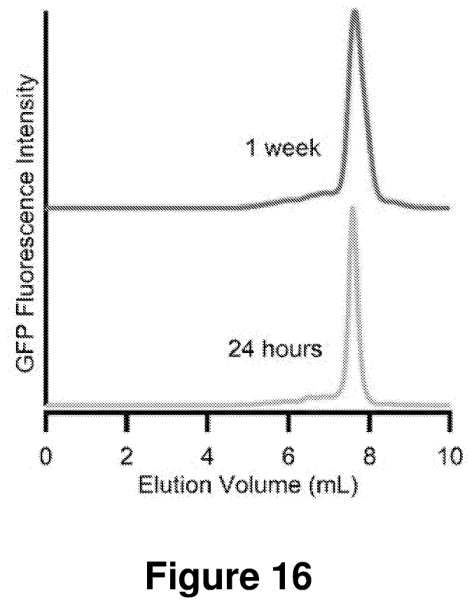

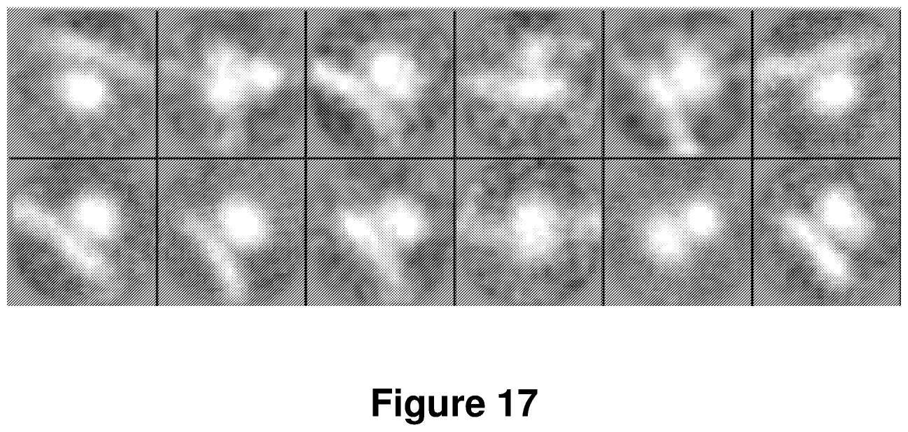

Chain-growth polymerization. Having shown that DNA can mediate the spontaneous polymerization of proteins resulting in product distributions consistent with a step-growth process, the overarching hypothesis of this work was next tested: that the underlying pathway of protein-monomer polymerization can be controlled by the secondary structure of the appended DNA sequence, which in turn controls the energy barrier to polymerization. First, H_{A} and H_{B} monomers were combined under identical conditions to those studied in the step-growth system, to test whether the hairpin DNA design impeded the spontaneous polymerization of monomers as desired. Indeed, SEC profiles were observed that were indistinguishable from the individual monomers, even after one week of incubation at room temperature (FIG. 15B, FIG. 16). Furthermore, the absence of any polymerized species was evident from cryoEM images (FIG. 15C). While the structure of the mGFP-hairpin monomers isn't immediately obvious upon inspection, 2D class averages of approximately 250 particles clearly show electron density corresponding to both mGFP and the hairpin appendage (FIG. 15C, inset, FIG. 17). Importantly, previously reported attempts to apply the hybridization chain reaction to control the association of proteins were unsuccessful due to the challenge of annealing hairpins conjugated to thermally unstable proteins. ⁴⁰ Here, however, this problem was circumvented by snap-cooling the hairpin DNA prior to the protein conjugation reaction described above.

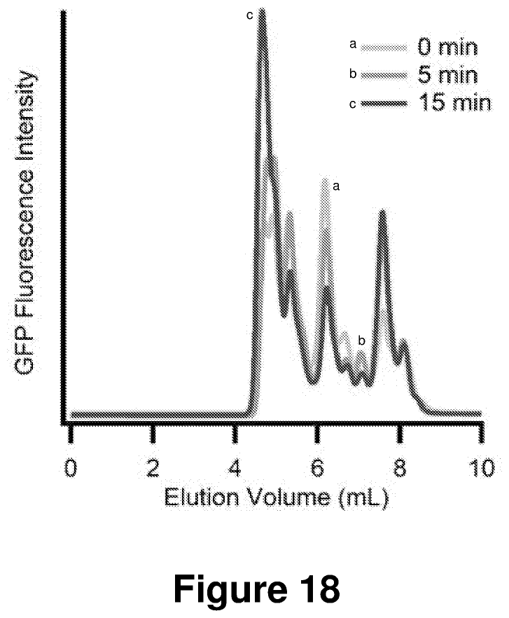

The addition of the initiator strand induces the polymerization of GFP-DNA monomers, as evidenced by SEC (FIG. 15E). Varying the equivalents of initiator strand with respect to monomer dramatically changes the molecular weight distribution of aggregates observed by SEC (FIG. 15E). Qualitatively, these chromatograms show that the molecular weight distribution decreases with increasing equivalents of initiator, with species below the exclusion limit of the column becoming more prominent at higher initiator concentrations, consistent with a chain-growth polymerization process. Cryo-EM analysis of these samples allowed this change to be quantified: a steady increase in both number and weight average degree of polymerization from 3.7 and 4.9, to 6.9 and 10.2 units was observed from 1 to 0.4 equivalents of initiator, respectively (FIG. 15D-G). Importantly, these images also reveal the presence of only linear products for all initiator concentrations tested, in stark contrast with the large population of cyclic products observed for the step-growth system. Since polymers growing via a chain-growth process contain only one single stranded “active end”, with the other end remaining fully duplexed with initiator, cyclization events are not kinetically accessible. This change in product distribution from a mixture of both cyclic and linear species, to exclusively linear, therefore reflects the change in polymer formation pathway. The initial rate of monomer consumption was also estimated via SEC, which increased with increasing initiator concentration, another key characteristic of chain-growth pathways at the molecular scale (FIG. 15G). Furthermore, the product distribution of the system could also be shifted by changes in the timing of initiator addition, similar to molecular polymerization techniques.⁴¹ When 1 equivalent of initiator was added in 5 aliquots over 25 or 75 minutes, an SEC profile with a significantly larger fraction of high molecular weight products was observed, with the percentage of species eluting with a retention volume below 5 mL increasing from 27%, to 31% and 43% of the overall integrated area of the mGFP fluorescence signal, respectively (FIG. 18). This suggests that directing protein polymerization via the hybridization chain reaction enables control over both molecular weight and polydispersity of the resulting protein polymers.

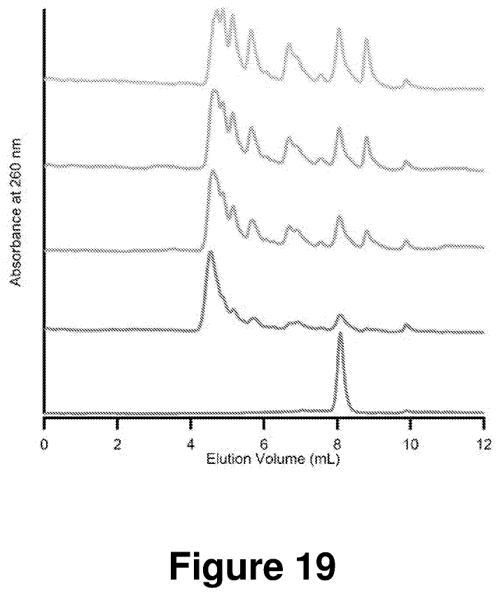

Ultimately this system displayed some important differences with an idealized chain-growth polymerization. In an ideal chain-growth reaction, the rate of initiation is fast relative to propagation and Mₙ=[M]₀/[I]. In this system, however, Mₙ is much greater than predicted from the [M]₀/[I], suggesting that the initiation reaction does not reach completion before monomer is depleted. In contrast with typical chain growth processes, for example atom transfer radical polymerization (ATRP),⁴² where the rate of initiation is much faster than the rate of propagation, the rate of initiation in this system is likely similar to the rate of propagation, owing to the identical chemical nature of these two reactions from a DNA perspective. In addition, with initiator concentrations below 0.6 equivalents, a decrease in conversion from approximately 90 to 74% was observed that persisted even after several weeks. These results were compared to the free DNA system polymerized under identical conditions and observed almost complete consumption of monomer (90%) for 0.4 equivalents of initiator, which suggested the incomplete conversion observed for low initiator concentrations is not a result of thermodynamics, but may be a mass-transfer or chain-end accessibility problem, which will be the subject of future investigations (FIG. 19).

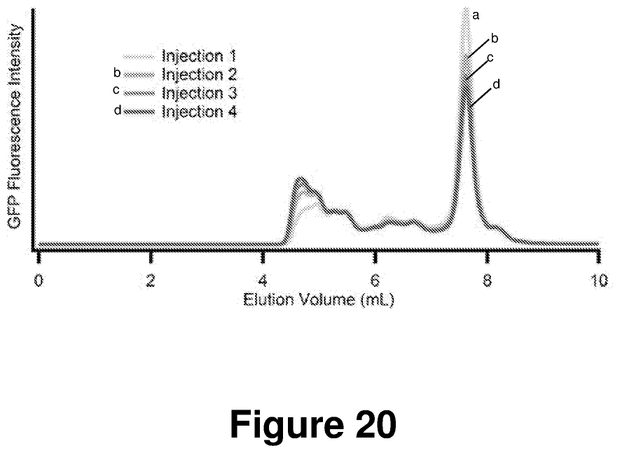

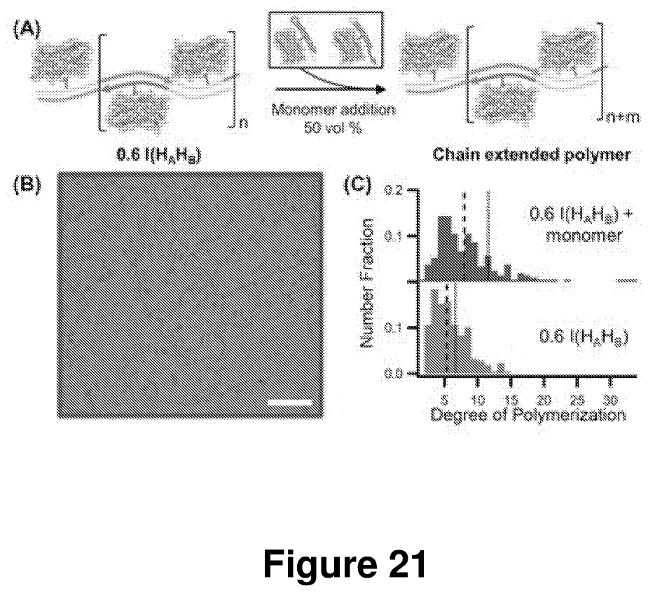

Chain extension. Certain classes of covalent and supramolecular chain-growth polymerizations display a living character, where chain termination events are absent. In these systems, because active chain ends persist indefinitely, the addition of fresh monomer to a sample of polymer results in the consumption of the monomer, and subsequent increase in molecular weight distribution of the polymer sample. The hybridization chain reaction employed herein has been proposed to possess a living polymerization character,²⁴ and based on the DNA sequences, no chain termination or combination events should be possible. Therefore, to test the living character of the chain-growth system, a polymerized solution of H_{A} and H_{B} was added with 0.6 equivalents of initiator to an equal volume of metastable monomer solution containing no initiator. Monitoring the monomer fraction in solution after the addition of the polymer, the consumption of the monomer over time was observed via SEC (FIGS. 3, 20), demonstrating that polymerization continues and suggesting chain extension. To characterize the change in molecular weight distribution after the addition of fresh monomer, cryo-EM analysis was conducted on this sample, which revealed a substantial increase in the number and weight average degree of polymerization from 5.4 to 7.3, and 6.7 to 13.6, respectively. This excludes the possibility that the monomer consumption observed via SEC is solely a result of excess initiator strands reacting with fresh monomer, and conclusively demonstrated that the DNA-mediated chain-growth polymerization of proteins reported herein possesses a living character.

Conclusion. The complexity observed in the assembly processes of proteins into highly intricate and functional polymeric architectures in nature has been unparalleled in the synthetic space. An initial step in this direction is reported herein by providing the first demonstration of designed protein polymerization pathway control. This work enables the realization of currently inaccessible protein architectures, including sequence-defined, multi-block, brush and branched protein polymer architectures that could represent important material targets for catalysis, sensing and tissue engineering applications, and pharmaceutical development. The work reported herein constitutes unprecedented control over the product distributions of protein polymers, and opens the door to systematically investigating and controlling their physical and chemical properties. Taken together, this study stands as a powerful demonstration of how DNA can be used to precisely tune the energy landscape, and thereby assembly pathways, of nanoscale building blocks, and will open the door to synthesizing entirely new classes of protein-based materials.

### REFERENCES

1. Pollard, T. D.; Borisy, G. G., Cellular Motility Driven by Assembly and Disassembly of Actin Filaments. Cell 2003, 112 (4), 453-465.
2. Artzi, L.; Bayer, E. A.; Moraïs, S., Cellulosomes: bacterial nanomachines for dismantling plant polysaccharides. Nat. Rev. Micro. 2016, 15, 83.
3. Desai, A.; Mitchison, T. J., Microtubule Polymerization Dynamics. Annu. Rev. Cell Dev. Biol. 1997, 13 (1), 83-117.
4. Nogales, E., Structural Insights into Microtubule Function. Ann. Rev. Biochem. 2000, 69 (1), 277-302.
5. Carlson, J. C. T.; Jena, S. S.; Flenniken, M.; Chou, T.-f.; Siegel, R. A.; Wagner, C. R., Chemically Controlled Self-Assembly of Protein Nanorings. J. Am. Chem. Soc. 2006, 128 (23), 7630-7638.
6. Gholami, Z.; Hanley, Q., Controlled Assembly of SNAP-PNA-Fluorophore Systems on DNA Templates To Produce Fluorescence Resonance Energy Transfer. Bioconjug. Chem. 2014, 25 (10), 1820-1828.
7. Brodin, J. D.; Ambroggio, X. I.; Tang, C.; Parent, K. N.; Baker, T. S.; Tezcan, F. A., Metal-directed, chemically tunable assembly of one-, two- and three-dimensional crystalline protein arrays. Nat. Chem. 2012, 4, 375.
8. Biswas, S.; Kinbara, K.; Oya, N.; Ishii, N.; Taguchi, H.; Aida, T., A Tubular Biocontainer: Metal lon-Induced 1D Assembly of a Molecularly Engineered Chaperonin. J. Am. Chem. Soc. 2009, 131 (22), 7556-7557.
9. Modica, J. A.; Lin, Y.; Mrksich, M., Synthesis of Cyclic Megamolecules. J. Am. Chem. Soc. 2018, 140 (20), 6391-6399.
10. Carothers, W. H., Polymerization. Chemical Reviews 1931, 8 (3), 353-426.
11. Grubbs, R. B.; Grubbs, R. H., 50th Anniversary Perspective: Living Polymerization-Emphasizing the Molecule in Macromolecules. Macromolecules 2017, 50 (18), 6979-6997.
12. Polymeropoulos, G.; Zapsas, G.; Ntetsikas, K.; Bilalis, P.; Gnanou, Y.; Hadjichristidis, N., 50th Anniversary Perspective: Polymers with Complex Architectures. Macromolecules 2017, 50 (4), 1253-1290.
13. Aida, T.; Meijer, E. W.; Stupp, S. I., Functional Supramolecular Polymers. Science 2012, 335 (6070), 813-817.
14. Ogi, S.; Sugiyasu, K.; Manna, S.; Samitsu, S.; Takeuchi, M., Living supramolecular polymerization realized through a biomimetic approach. Nat. Chem. 2014, 6, 188.
15. Kang, J.; Miyajima, D.; Mori, T.; Inoue, Y.; Itoh, Y.; Aida, T., A rational strategy for the realization of chain-growth supramolecular polymerization. Science 2015, 347 (6222), 646-651.
16. Sijbesma, R. P.; Beijer, F. H.; Brunsveld, L.; Folmer, B. J. B.; Hirschberg, J. H. K. K.; Lange, R. F. M.; Lowe, J. K. L.; Meijer, E. W., Reversible Polymers Formed from Self-Complementary Monomers Using Quadruple Hydrogen Bonding. Science 1997, 278 (5343), 1601-1604.
17. McMillan, J. R.; Mirkin, C. A., DNA-Functionalized, Bivalent Proteins. J. Am. Chem. Soc. 2018, 140 (22), 6776-6779.
18. Brodin, J. D.; Auyeung, E.; Mirkin, C. A., DNA-mediated engineering of multicomponent enzyme crystals. Proc. Natl. Acad. Sci. U.S.A 2015, 112 (15), 4564-4569.
19. Kashiwagi, D.; Sim, S.; Niwa, T.; Taguchi, H.; Aida, T., Protein Nanotube Selectively Cleavable with DNA: Supramolecular Polymerization of “DNA-Appended Molecular Chaperones”. J. Am. Chem. Soc. 2018, 140 (1), 26-29.
20. Mirkin, C. A.; Letsinger, R. L.; Mucic, R. C.; Storhoff, J. J., A DNA-based method for rationally assembling nanoparticles into macroscopic materials. Nature 1996, 382 (6592), 607-609.
21. Macfarlane, R. J.; Lee, B.; Jones, M. R.; Harris, N.; Schatz, G. C.; Mirkin, C. A., Nanoparticle Superlattice Engineering with DNA. Science 2011, 334 (6053), 204-208.
22. Jones, M. R.; Seeman, N. C.; Mirkin, C. A., Programmable materials and the nature of the DNA bond. Science 2015, 347 (6224).
23. Hayes, O. G.; McMillan, J. R.; Lee, B.; Mirkin, C. A., DNA-Encoded Protein Janus Nanoparticles. J. Am. Chem. Soc. 2018, 140 (29), 9269-9274.
24. Dirks, R. M.; Pierce, N. A., Triggered amplification by hybridization chain reaction. PNAS 2004, 101 (43), 15275-15278.
25. Khoshouei, M.; Radjainia, M.; Baumeister, W.; Danev, R., Cryo-EM structure of haemoglobin at 3.2 Å determined with the Volta phase plate. Nat. Commun. 2017, 8, 16099.
26. Danev, R.; Buijsse, B.; Khoshouei, M.; Plitzko, J. M.; Baumeister, W., Volta potential phase plate for in-focus phase contrast transmission electron microscopy. PNAS 2014, 111 (44), 15635-15640.
27. Chua, E. Y. D.; Vogirala, V. K.; Inian, O.; Wong, A. S. W.; Nordenskiöld, L.; Plitzko, J. M.; Danev, R.; Sandin, S., 3.9 Å structure of the nucleosome core particle determined by phase-plate cryo-EM. Nucleic Acids Res. 2016, 44 (17), 8013-8019.
28. Usov, I.; Mezzenga, R., FiberApp: An Open-Source Software for Tracking and Analyzing Polymers, Filaments, Biomacromolecules, and Fibrous Objects. Macromolecules 2015, 48 (5), 1269-1280.
29. Xu, J.; Fogleman, E. A.; Craig, S. L., Structure and Properties of DNA-Based Reversible Polymers. Macromolecules 2004, 37 (5), 1863-1870.
30. Baumann, C. G.; Smith, S. B.; Bloomfield, V. A.; Bustamante, C., lonic effects on the elasticity of single DNA molecules. PNAS 1997, 94 (12), 6185-6190.
31. Marko, J. F.; Siggia, E. D., Stretching DNA. Macromolecules 1995, 28 (26), 8759-8770.
32. Bustamante, C.; Marko, J.; Siggia, E.; Smith, S., Entropic elasticity of lambda-phage DNA. Science 1994, 265 (5178), 1599-1600.
33. Cloutier, T. E.; Widom, J., Spontaneous Sharp Bending of Double-Stranded DNA. Mol. Cell 2004, 14 (3), 355-362.
34. Vafabakhsh, R.; Ha, T., Extreme Bendability of DNA Less than 100 Base Pairs Long Revealed by Single-Molecule Cyclization. Science 2012, 337 (6098), 1097-1101.
35. Du, Q.; Kotlyar, A.; Vologodskii, A., Kinking the double helix by bending deformation. Nucleic Acids Res. 2008, 36 (4), 1120-1128.
36. Wolters, M.; Wittig, B., Construction of a 42 base pair double stranded DNA microcircle. Nucleic Acids Res. 1989, 17 (13), 5163-5172.
37. Yan, J.; Marko, J. F., Localized Single-Stranded Bubble Mechanism for Cyclization of Short Double Helix DNA. Phys. Rev. Lett. 2004, 93 (10), 108108.
38. Protozanova, E.; Yakovchuk, P.; Frank-Kamenetskii, M. D., Stacked-Unstacked Equilibrium at the Nick Site of DNA. J. Mol. Bio. 2004, 342 (3), 775-785.
39. Demurtas, D.; Amzallag, A.; Rawdon, E. J.; Maddocks, J. H.; Dubochet, J.; Stasiak, A., Bending modes of DNA directly addressed by cryo-electron microscopy of DNA minicircles. Nucleic Acids Res. 2009, 37 (9), 2882-2893.
40. Chen, R. P.; Blackstock, D.; Sun, Q.; Chen, W., Dynamic protein assembly by programmable DNA strand displacement. Nat. Chem. 2018, 10 (4), 474-481.
41. Gentekos, D. T.; Dupuis, L. N.; Fors, B. P., Beyond Dispersity: Deterministic Control of Polymer Molecular Weight Distribution. J. Am. Chem. Soc. 2016, 138 (6), 1848-1851.
42. Wang, J.-S.; Matyjaszewski, K., Controlled/“living” radical polymerization. atom transfer radical polymerization in the presence of transition-metal complexes. J. Am. Chem. Soc. 1995, 117 (20), 5614-5615.

1. A method of making a protein polymer comprising contacting:
(a) a first protein monomer comprising a first protein to which a first oligonucleotide is attached, the first oligonucleotide comprising a first domain (V) and a second domain (W); and
(b) a second protein monomer comprising a second protein to which a second oligonucleotide is attached, the second oligonucleotide comprising a first domain (V′) and a second domain (W′),
wherein (i) V is sufficiently complementary to V′ to hybridize under appropriate conditions and (ii) W is sufficiently complementary to W′ to hybridize under appropriate conditions, and wherein the contacting results in V hybridizing to V′,
thereby making the protein polymer.

2. (canceled)

3. (canceled)

4. The method of claim 1, wherein the first protein and the second protein are different.

5. (canceled)

6. The method of claim 1, wherein the first oligonucleotide is attached to the first protein via a lysine or cysteine on the surface of the first protein.

7. The method of claim 1, wherein the first oligonucleotide is DNA, RNA, a combination thereof, or a modified form thereof.

8. (canceled)

9. (canceled)

10. The method of claim 1, wherein
the second oligonucleotide is attached to the second protein via a lysine or cysteine on the surface of the second protein.

11. The method of claim 1, wherein
the second oligonucleotide is DNA, RNA, a combination thereof, or a modified form thereof.

12. (canceled)

13. (canceled)

14. The method of claim 1, wherein the protein polymer is a hydrogel or a therapeutic.

15. The method of claim 14, wherein the therapeutic is an antibody, a cell penetrating peptide, a viral capsid, an intrinsically disordered protein, a lectin, or a membrane protein.

16. A method of making a protein polymer comprising contacting:
(a) a first protein monomer comprising a first protein to which a first oligonucleotide is attached, the first oligonucleotide comprising a first domain (X), a second domain (Y′), a third domain (Z), and a fourth domain (Y), wherein Y is sufficiently complementary to Y′ to hybridize under appropriate conditions to produce a first hairpin structure;
(b) a second protein monomer comprising a second protein to which a second oligonucleotide is attached, the second oligonucleotide comprising a first domain (Y), a second domain (X′), a third domain (Y′), and a fourth domain (Z′), wherein Y is sufficiently complementary to Y′ to hybridize under appropriate conditions to produce a second hairpin structure; and
(c) an initiator oligonucleotide comprising a first domain (Y) and a second domain (X′);
wherein the contacting results in (i) X′ of the initiator oligonucleotide hybridizing to X of the first oligonucleotide and Y of the initiator oligonucleotide displacing Y of the first oligonucleotide, thereby opening the first hairpin structure and (ii) Z′ of the second oligonucleotide hybridizing to Z of the first oligonucleotide thereby opening the second hairpin structure, and
thereby making the protein polymer.

17. (canceled)

18. The method of claim 16, wherein the first protein and the second protein are different.

19. (canceled)

20. The method of claim 16, wherein the first oligonucleotide is attached to the first protein via a lysine or cysteine on the surface of the first protein.

21. The method of claim 16, wherein the first oligonucleotide is DNA, RNA, a combination thereof, or a modified form thereof.

22. (canceled)

23. (canceled)

24. (canceled)

25. (canceled)

26. The method of claim 16, wherein the second oligonucleotide is attached to the second protein via a lysine or cysteine on the surface of the second protein.

27. The method of claim 16, wherein the second oligonucleotide is DNA, RNA, a combination thereof, or a modified form thereof.

28. (canceled)

29. (canceled)

30. (canceled)

31. (canceled)

32. The method of claim 16, wherein the protein polymer is a hydrogel or a therapeutic.

33. The method of claim 32, wherein the therapeutic is an antibody, a cell penetrating peptide, a viral capsid, an intrinsically disordered protein, a lectin, or a membrane protein.

34. The method of claim 16, further comprising adding a third protein monomer comprising a third protein to which a third oligonucleotide is attached, the third oligonucleotide comprising a first domain (X), a second domain (Y′), a third domain (Z), and a fourth domain (Y), wherein Y is sufficiently complementary to Y′ to hybridize under appropriate conditions to produce a third hairpin structure.

35. (canceled)

36. (canceled)

37. The method of claim 16, further comprising adding a fourth protein monomer comprising a fourth protein to which a fourth oligonucleotide is attached, the fourth oligonucleotide comprising a first domain (Y), a second domain (X′), a third domain (Y′), and a fourth domain (Z′), wherein Y is sufficiently complementary to Y′ to hybridize under appropriate conditions to produce a fourth hairpin structure.

38. (canceled)

39. (canceled)

40. A method of treating a subject in need thereof comprising administering the protein polymer of claim 16 to the subject.

41. A composition comprising the protein polymer of claim 16 and a physiologically acceptable carrier.


SEQUENCE LISTING (5 sequences)

SEQ ID NO: 1 - DNA, 48 bp, synthetic construct
  misc_feature 1..48: note = Synthetic Polynucleotide
  modified_base 40: mod_base = OTHER; note = Amino group (NH2)
  source 1..48: mol_type = other DNA
ttaacccacgccgaatcctagactcaaagtagtctaggattcggcgtg

SEQ ID NO: 2 - DNA, 46 bp, synthetic construct
  misc_feature 1..46: note = Synthetic Polynucleotide
  modified_base 11: mod_base = OTHER; note = Amino group (NH2)
  source 1..46: mol_type = other DNA
agtctaggattcggcgtgggttaacacgccgaaccagactactttg

SEQ ID NO: 3 - DNA, 24 bp, synthetic construct
  misc_feature 1..24: note = Synthetic Polynucleotide
  source 1..24: mol_type = other DNA
agtctaggattcggcgtgggttaa

SEQ ID NO: 4 - DNA, 48 bp, synthetic construct
  misc_feature 1..48: note = Synthetic Polynucleotide
  modified_base 40: mod_base = OTHER; note = Amino group (NH2)
  source 1..48: mol_type = other DNA
ttagtcgtctctcatcatgtgttacaaagtagtctaggattcggcgtg

SEQ ID NO: 5 - DNA, 48 bp, synthetic construct
  misc_feature 1..48: note = Synthetic Polynucleotide
  modified_base 11: mod_base = OTHER; note = Amino group (NH2)
  source 1..48: mol_type = other DNA
taacacatgatgagagacgactaacacgccgaatcctagactactttg